(19) United States
(12) Patent Application Publication
(10) Pub. No.: US 2011/0165645 A1
(43) Pub. Date: Jul. 7, 2011

(54) Methods Using Ion Exchange and Gel Filtration Chromatography for Poxvirus Purification

(76) Inventor: Yelin Xiong, Richmond Hill (CA)

(21) Appl. No.: 12/867,047

(22) PCT Filed: Feb. 12, 2009
(86) PCT No.: PCT/CA2009/000141

§ 371 Date: Mar. 21, 2011

Related U.S. Application Data
(60) Provisional application No. 61/065,484, filed on Feb. 12, 2008.

(51) Int. Cl. C12N 7/02; C12N 13/00

(52) U.S. Cl. 435/173.9; 435/239

(57) ABSTRACT

Provided herein are methods for purifying poxviruses using one or more chromatographic steps including, but not limited to, gel filtration and/or ion exchange chromatography.

### RELATED APPLICATIONS

This application claims priority to U.S. Ser. No. 61/065,484 filed Feb. 12, 2008.

### FIELD OF STUDY

This document describes methods for isolating vectors such as poxviral vectors, and in avipox (e.g., canarypox, ALVAC) vectors.

### BACKGROUND INFORMATION

Various types of chromatographic procedures have been utilized to purify viruses. Anion exchange chromatography is the most common chromatography column purification method used for virus purification. It has been used to purify a variety of viruses including HIV-1 (Prior et al., 1995; 1996), Sendai virus (Eveleth et al, 2000), recombinant adeno-associated virus (Huyghe et al., 1995; Kaludov et al., 2002), and lentivirus (Yamada et al., 2003). Cation exchange chromatography has also been utilized (Gao et al, 2000). Size exclusion chromatography (SEC) has proved to be a potential gental method for virus purification (Braas et al., 1996). Recombinant adenoviruses and adeno-associated viruses have been isolated using hydrophobic interaction chromatography (HIC) has been used for recombinant adenovirus or recombinant adeno-associated virus purification, either in the binding and elution mode (Huyghe et al., 1995), or in the flow-through mode (Snyder and Flotte, 2002). And ceramic hydroxyapaptite (CHT) has been was used successfully to purify Moloney murine leukaemia virus (Kuiper et al., 2002). Affinity purification has also been shown to be useful for purifying many types of viruses, especially those with lipid envelopes (Millipore Data Sheet; O'Neil and Balkovic, 1993; O'Neil and Balkovic, 1993; Tamayose et al., 1996). Heparin-based affinity chromatography resin has been used for purification of viruses, including recombinant adeno-associated virus (Clark et al., 1999; Zolotukhin et al., 1999; Auricchio et al., 2001; Summerford and Samulski, 1999) and Herpes Simplex Virus (O'Keeffe et al., 1999). There is still a need in the art for additional improved purification methods. Toward this end, improved processes for purifying poxviruses are provided herein.

### SUMMARY

Provided herein are methods for purifying poxviruses using one or more chromatographic steps including, but not limited to, gel filtration and/or ion exchange chromatography. In certain embodiments, the poxvirus is an avipox virus (e.g., canarypox, ALVAC).

### BRIEF DESCRIPTION OF THE FIGURES

FIG. 1. Diagram of 10 L scale ANX ion exchange batch adsorption.

FIG. 2. Optimal operating TMP for various operating shear rates.

FIG. 3. TFF performance under different TMP and shear rates (lumen ID 0.5 mm).

FIG. 4. TFF performance under different TMP and shear rates (lumen ID 1 mm).

FIG. 5. TFF performance under different TMP and shear rates-Concentrating clarified ALVAC/CEFs.

### DETAILED DESCRIPTION

Provided herein are methods for purifying recombinant or “wild-type” poxvirus vectors (e.g., poxviral particles, virions) comprising subjecting a crude poxvirus preparation (or a derivative thereof, such as a semi-purified poxvirus preparation) to ion exchange chromatography to produce a poxvirus preparation with reduced levels of contaminants. A poxvirus preparation is one in which intact poxvirus particles or virions (which may simply be referred to as poxvirus) are present. The poxvirus particles or virions may be, for example, wild-type, attenuated, non-recombinant, or recombinant. Contaminants (e.g., non-poxviral components) are components other than intact poxviral particles or virions. Contaminants are typically biological (e.g., not including buffers, excipients and the like) and may include, for example, non-vector DNA and/or RNA, free vector DNA and/or RNA, other RNA and/or DNA, non-vector peptides or proteins, other free peptide or proteins, and the like. In some embodiments, the process results in the removal of up to approximately or specifically 80% to 99% of the total protein (including peptides) and/or total nucleic acid (e.g., DNA, RNA) contaminants present in the crude poxvirus. In some embodiments, up to approximately or specifically 80%, 85%, 90%, 95%, or 99% of the total protein (including peptides) and/or total nucleic acid (e.g., DNA, RNA) contaminants present in the crude poxvirus preparation are removed.

In one embodiment, the method comprises subjecting a crude poxvirus preparation to ion exchange chromatography to produce a poxvirus preparation that is substantially free of contaminants (a “substantially purified poxvirus preparation”). A substantially purified preparation is substantially free of contaminants where those contaminants total less than approximately or specifically 20 to 30% by weight (excluding carriers, excipients and the like) of the preparation. In certain embodiments, a preparation is substantially purified where the contaminants total less than approximately or specifically 20-30%, 20-22.5%, 22.5-25%, 25-27.5%, or 30% by weight in the preparation as a whole, or relative to the poxvirus per se. A preparation may also be considered substantially purified where at least approximately or specifically 80% to 89% of the contaminants present in the crude poxvirus preparation (that is not part of a poxvirus) have been removed from the preparation.

In one embodiment, the method comprises subjecting a crude poxvirus preparation to ion exchange chromatography to produce a poxvirus preparation that is essentially free of contaminants (an “essentially purified poxvirus preparation”). An essentially purified preparation is essentially free of contaminants where those contaminants total less than approximately or specifically 10 to 20% by weight (excluding carriers, excipients and the like) of the preparation. In certain cases, the contaminants of an essentially purified preparation total less than approximately or specifically 10-20%, 10-12.5%, 12.5-15%, 15-17.5% or 20% by weight in the preparation as a whole, or relative to the poxvirus per se. A preparation may also be considered essentially purified where at least approximately or specifically 90% to 95% of the contaminants have been removed from the preparation.

In one embodiment, the method comprises subjecting a crude poxvirus preparation to the purification process to produce a poxvirus preparation that is free of contaminants (a “purified poxvirus preparation”). A purified poxvirus preparation is free of contaminants where the contaminants total less than approximately or specifically 0 to 10% by weight (excluding carriers, excipients and the like) of the preparation. In certain embodiments, a preparation is free of contaminants where those contaminants total less than approximately or specifically 0-10%, 7.5-10%, 5-7.5%, 2.5-5%, or 1% by weight in the preparation as a whole, or relative to the poxvirus per se. A preparation may also be considered purified where at least approximately or specifically 95% to 99%, or 100% of the contaminants present in the crude poxvirus preparation (that is not part of a poxvirus) are removed from the preparation.

Also provided is method for purifying a poxvirus comprising contacting a sample (e.g., a cell lysate) containing the poxvirus and at least one contaminant with an ion exchange chromatography matrix under conditions providing selective interaction of the poxvirus with the matrix with respect to contaminants and eluting the poxvirus from the matrix. “Selective interaction” may be achieved by any means such as, for example, exposing the sample to the matrix under conditions allowing the poxvirus to bind the matrix more efficiently than contaminants or through utilization of washing and/or elution conditions that allow the poxvirus to remain bound to the matrix and cause contaminants to be released from the matrix. In certain of these methods, a sample (e.g., a cell lysate) containing poxvirus and contaminants may be contacted with an ion exchange matrix that selectively interacts with the poxvirus relative to contaminants and eluting the bound poxvirus from the matrix. Another method for isolating a poxvirus from a partially purified sample (e.g., a cell lysate, a concentrated cell lysate) includes: (a) providing a partially purified sample containing a poxvirus; (b) contacting said partially purified sample with a solid support comprising an ion-exchange matrix under conditions in which the poxvirus binds to the matrix; and (c) eluting the bound poxvirus from the solid support.

A crude poxvirus preparation (e.g., a cell lysate or concentrated cell lysate) may be partially purified prior to further purification to provide a partially purified sample. The partially purified sample may then be subjected to further purification. Where the poxvirus is cultured in cells and a partially purified preparation is desired, the following process may be used: harvesting the poxvirus-containing cells; disrupting the cells by, for example, lysing the cells by enzymatic (e.g., trypsin and/or nucleases) or other means, to produce a crude poxvirus preparation; optionally clarifying the crude preparation by, for example, centrifugation or tangential flow filtration (TFF); submitting the crude poxvirus preparation to a purification step such as gel filtration to produce a semi-purified poxvirus preparation; and, submitting the semi-purified poxvirus preparation to further purification using, for instance, ion exchange chromatography to produce a substantially purified, essentially purified, or purified poxvirus preparation. The crude poxvirus preparation and the semi-purified poxvirus preparation typically may each contain contaminants totaling more than approximately or specifically 30% by weight (excluding carriers, excipients and the like) of the preparation. Typically, the semi-purified poxvirus preparation contains less contaminants than the crude poxvirus preparation. Other means of purification may also be included to produce a substantially purified, essentially purified, or purified poxvirus preparation.

Many suitable gel filtration matrices (also termed gel filtration resins) are available to one of skill in the art. Such resins include, for example, Sephacryl® (e.g., S-100 HR, S-200 HR, S-300 HR, S-400 HR), Sephadex® (e.g., Lipophilic (hydroxyalkoxypropyl-dextran, Type I, Type VI, or Type IX), G-10, G-15, G-25, G-50, G-75, G-100), Sepharose® (e.g., 6B, CL-6B, 4B, CL-4B, 2B, CL-2B), Superdex® (e.g., 30, 75, 200), Superose® (e.g., 12, 6), Toyopearl® HW (e.g., HW-40, HW-50, HW-55, HW-65, HW-75), Ultrogel® (e.g., Matrix A, AcA). Preferred gel filtration matrices may be Sepharose 4 Fast Flow or Sepharose 6 Fast Flow. Gel filtration matrices may be equilibrated as is known in the art. For example, as shown herein for the purification of poxviruses, a Tris-HCl buffer (e.g., 5 mM, 10 mM, 15 mM, 20 mM) at a pH of between approximately 7.0-9.0 may be suitable. In certain embodiments, a pH of approximately 7.0, 7.5, 8.0. 8.5, or 9.0 may be preferred. In certain other embodiments, a pH of approximately 9.0 may preferred. The use of other gel filtration matrices and buffer systems are known in the art and may be suitable in carrying out the methods described herein.

Many suitable ion exchange chromatography matrices (also termed ion exchange resins) are available to one of skill in the art. The ion exchange matrix may be selected from any of those available such as, for example, strong anion exchanger, a weak anion exchanger, a strong cation exchanger, and a weak cation exchanger. Exemplary matrices include, for example, Q Sepharose™ Fast Flow, SP Sepharose™ Fast Flow, CM Sepharose™ Fast Flow, DEAE Sepharose™ Fast Flow, and ANX Sepharose™ 4 Fast Flow, among others. A preferred media is ANX Sepharose 4 Fast Flow resin which may be equilibrated with, for example, a Tris-HCl buffer (e.g., 5 mM, 10 mM, 15 mM, 20 mM) at a pH of between approximately 7.0-9.0. Preferably, the buffer may be 10 mM Tris-HCl at a pH of approximately 7.0, 7.5, 8.0, 8.5, or 9.0. The use of other ion exchange matrices and buffer systems is known in the art and may be suitable in carrying out the methods described herein.

In certain of the methods described herein, elution is carried out by contacting the poxvirus bound to the ion exchange matrix with an elution buffer. As described above, in certain embodiments, it is preferred that the matrix and/or elution system be selective for poxviruses. For example, one may utilize a preliminary elution step removes the majority of the contaminants from the resin, and a following elution step to remove the poxviral particles from the matrix. One may also utilize, as an alternative or in combination with the previously described elution step or steps, an elution step that primarily removes the majority of the poxviral particles from the matrix while leaving the contaminants bound to the matrix. A washing step may also be utilized to remove the contaminants such that the majority of the material bound to the matrix are poxviral components. In such cases, a single elution step may be utilized to remove bound poxviral particles from the resin. Typically, a salt solution is used as the elution buffer. Any suitable salt may be utilized in the elution buffer. In certain embodiments, sodium chloride (NaCl) may be used. And in some embodiments, a high salt buffer may be utilized. A high salt buffer is typically approximately or specifically 300 mM, 600 mM or 1 M salt (e.g., NaCl). For instance, elution may be performed in a suitable buffer containing approximately or specifically 300 mM, 600 mM or 1 M NaCl. Any suitable buffer may be used such as, for example, a Tris CL (e.g., 5, 10, 15 or 20 mM) buffer. In certain embodiments, it is preferred that elution is performed using a buffer such as Tris at a pH of approximately or specifically 7.0, 7.5, 8.0. 8.5, or 9.0 containing a high concentration of salt (e.g., 300 mM, 600 mM, or 1M). The use of other elution buffers is known in the art and may be suitable in carrying out the methods described herein.

A partially purified sample such as a cell lysate may be subjected to any of several procedures, including, for example, ammonium sulfate precipitation, dialysis, size-exclusion fractionation, density gradient fractionation, sucrose cushion ultracentrifugation, or exposure to an enzyme. Exemplary enzymes include, for example, a protease (e.g., trypsin), an endonuclease (e.g., benzonase), or other enzyme. Any of these procedures may be used prior to any other procedure, alone or in combination, and may be used prior to subjecting the sample to ion exchange chromatography to produce a substantially purified, essentially purified, or purified poxviral preparation.

The methods described herein may be utilized to isolate viruses, including but not limited to poxviruses (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). Exemplary poxviruses are vaccinia and derivatives thereof such as NYVAC and Modified Ankara Virus (MVA), avipox, fowlpox, canarypox, ALVAC, and ALVAC(2), among others. The poxviruses may be recombinant, meaning that the poxvirus genome contains exogenous nucleic acid sequence therein. Recombinant poxviruses may take the form of recombinant poxviral particles (alternatively referred to as recombinant virions), for example.

NYVAC (vP866) was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R); hemorrhagic region (u; B13R+B14R); A type inclusion body region (ATI; A26L); hemagglutinin gene (HA; A56R); host range gene region (C7L-K1L); and, large subunit, ribonucleotide reductase (14 L). NYVAC is a genetically engineered vaccinia virus strain that was generated by the specific deletion of eighteen open reading frames encoding gene products associated with virulence and host range. NYVAC has been show to be useful for expressing tumor antigens (see, for example, U.S. Pat. No. 6,265,189). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively.

Modified virus Ankara (MVA) has been previously described in, for example, U.S. Pat. Nos. 5,185,146 and 6,440,422; Sutter, et al. (B. Dev. Biol. Stand. Basel, Karger 84:195-200 (1995)); Antoine, et al. (Virology 244: 365-396, 1998); Sutter et al. (Proc. Natl. Acad. Sci. USA 89: 10847-10851, 1992); Meyer et al. (J. Gen. Virol. 72: 1031-1038, 1991); Mahnel, ett al. (Berlin Munch. Tierarztl. Wochenschr. 107: 253-256, 1994); Mayr et al. (Zbl. Bakt. Hyg. I, Abt. Org. B 167: 375-390 (1987); and, Stickl et al. (Dtsch. med. Wschr. 99: 2386-2392 (1974)). MVA is available from the ATCC under accession numbers VR-1508 and VR-1566.

ALVAC-based recombinant viruses (i.e., ALVAC-1 and ALVAC-2) may also be purified using the methods described herein (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

TROVAC viruses may also be purified using the methods described herein. TROVAC refers to an attenuated fowlpox that was a plaque-cloned isolate derived from the FP-1 vaccine strain of fowlpoxvirus which is licensed for vaccination of 1 day old chicks. TROVAC was likewise deposited under the terms of the Budapest Treaty with the ATCC, accession number 2553.

Pharmaceutical compositions containing viruses purified by the methods described herein are also provided herein. A suitable pharmaceutical composition typically may include at least a virus and a pharmaceutically acceptable carrier and/or excipient (e.g., which are not considered contaminants). The term “pharmaceutically acceptable carrier” as used herein refers to one or more formulation materials suitable for accomplishing or enhancing the delivery of agent described herein. The formulation may include a buffer, a salt, a sugar, and/or similar compounds as are known in the art. Suitable compositions may include liquid preparations such as sterile suspensions, syrups, emulsions, or elixirs prepared as sterile for parental, subcutaneous, intradermal, intramuscular or intravenous administration. In addition, the compositions can be co-administered or sequentially administered with agents. A suitable daily dose for a human or other mammal may vary widely depending on the type of virus being administered, the condition of the patient and other factors, but may be determined using routine methods.

A kit comprising the reagents for purifying viruses using the methods described herein is also provided. The kit may include, for example, buffers, filters and the like such that the skilled artisan may carry out the methods described herein. Additionally, the kit may include instructions for carrying out the methods described herein.

Abbreviations used in this document include the following: CPE: Cytopathic effect; CCID₅₀: Cell culture infectious dose 50%; CEF: Chicken embryo fibroblasts; CHT: Ceramic Hydroxyapatite; CIM: Convective Interaction media; CV: Column Volume; EBA: Expanded bed adsorption; EB14 cell line: A stable diploid cell line derived by VIVALIS France from chicken embryonic stem cell; EDTA: Ethylenediamine Tetraacetic acid; EEV: Extracellular enveloped virus; ELISA: Enzyme-Linked Immunosorbent Assay; FBS: Fetal bovine serum; FF: Fast flow; G: Centrifugation unit; GEQ: Genomic equivalence; IMV: Intracellular mature virus; LMH: Litre per square meter per hour; MOI: Multiplicity of infectivity; PBS: Phosphate-buffered saline; QT35: Chemically-induced fibrosarcomas from Japanese quail; qPCR: Quantitative polymerase chain reaction; RT: Room Temperature; TFF: Tangential flow filtration; TMP: Transmembrane pressure; WFI: Water for Injection

Cytopathic effect (CPE) is defined as the observation of morphological changes in cell structure such as cell rounding and detachment from the substrate, cell lysis, syncytium formation, and inclusion body formation resulting from virus infection. CCID₅₀ refers to the dilution of a virus required to infect 50% of a given batch of inoculated cell culture. The assay relies on the presence and detection of cytocidal virus particles. Host cells are grown in confluent healthy monolayers in a 96-well plate, to which aliquots of virus dilutions are added. The virus replicates and the progeny virions are released to infect healthy cells during incubation. The CPE is allowed to develop over a period of time, and wells are scored for the presence or absence of CPE. The “titre” of a viral suspension, expressed in infectious units per unit volume, is an estimate of the number of viral particles in a suspension that are able to produce a focus of infection or cytopathic effects under defined conditions. Poxvirus titres will vary with the type of cells used, methods of infection, and conditions of incubation. “GEQ” or genomic equivalence indicates that 1 genomic equivalence equal to 0.3 femtogram of DNA

A better understanding of the present invention and of its many advantages will be had from the following examples, given by way of illustration.

### EXAMPLES

The methods described herein are useful for purifying viruses such as poxviruses. A chromatography-based purification process for preparing compositions containing avipox viruses such as ALVAC with reduced levels of non-avipox DNA to meet regulatory requirements for vaccine safety, consistency and potency. Described below are materials, optimization experiments, and several exemplary methods for purifying viruses.

### I. Materials

Buffers used in these Examples include 10 mM Tris-HCl buffer, pH 7.4; 10 mM Tris-HCl buffer, pH 9.0; 10 mM Tris-HCl/1 M NaCl buffer, pH 7.4; 10 mM Tris-HCl/1 M NaCl buffer, pH 9.0. Other reagents utilized include 0.5M MgCl₂, 1M EDTA, Benzonase Endonuclease (EM Industries, Inc. Cat# 1.01694.0002 and 1.1697.0002), ALVAC-HIV (vCP1521)/EB14 harvests, ALVAC melanoma (vCP2264)/CEFs harvests, Trovax/chick embryonic fibroblasts (CEFs) and Trovax/duck cell lines (Cell & Viral Platform, AvP Canada). Chromatographic matrices utilized herein include Sepharose 4 FF weak anion exchanger (e.g., ANX Sepharose 4 FF (GE Healthcare, Cat# 17-1287-01 and 171287-04)), Sepharose 4 FF (GE Healthcare, Cat# 17-0149-01 and 17-0149-05), and Sepharose 6 FF (GE Healthcare, Cat# 17-0159-01).

The following represents a non-exhaustive list of equipment utilized in methods described below: AKTA Explorer, Unicorn software, GE Healthcare; BPG chromatography column 100/500, GE Healthcare; centrifuge (Jouan KR422, equipment #CEN1122 RSM 1167); Easy Load II Masterflex pump (Cole-Parmer Instrument Company, Model 77200-062, and Model 7529-10); Freezer, minus 70° C. (Sanyo, BIF0309); Profile star 5 μm depth filter (PALL, cat# BYA050P6); Profile star 3 μm depth filter (PALL, cat# BYA030P6); silicone tubing ( 3/16″ and ⅜″, Tygon, Cat# ABWO013); Virsonic 600 ultrasonic cell disrupter (sonicator); Misonix Flocell continuous flow chamber; TFF cartridge (GE Healthcare, Model # UPF-500-C-3×2MA); autoclave (Kuhlman, KG2119), Millipore polygard CN opticap XL5 depth filter (cat# KN1HA05HH1); incubator (SANYO, ID#2264, set at 38±1° C.); and, water bath (Polyscience, model#G-560).

### II. Methods

### A. Exemplary Method

The purification process described herein is useful for purifying pox virus-based vaccines. Such poxviruses include but are not limited to the ALVAC virus and derivatives thereof such as ALVAC-2. In general, the process includes the following steps:
1. Obtain a poxvirus harvest from a sample produced in cells using, for example, a bioreactor and concentrate (i.e., 10-fold) the harvest by centrifugation;
2. Release intracellular poxvirus by an appropriate method such as cell disruption by direct sonication to produce a crude poxvirus preparation;
3. Clarify the crude poxvirus preparation using, for example, sequential filtration with 5 μm and 3 μm depth filters;
4. Degrade free DNA present within the clarified crude poxvirus preparation using a reagent such as benzonase nuclease;
5. Produce a semi-purified poxvirus preparation by gel filtration using an appropriate chromatographic matrix and buffer system such as Sepharose 4 FF/6FF;
6. Purify a substantially purified, essentially purified, or purified poxvirus preparation using an appropriate ion exchange matrix such as Sepharose 4 FF (ANX); and,
7. Concentrate and exchange buffers by filtration (i.e., tangential flow filtration).

A particular embodiment of this method is described below. As shown therein, a purified poxvirus preparation (ALVAC) was successfully isolated from a poxvirus harvest.

### B. Purification of an ALVAC-HIV Vector

1. Obtain Poxvirus Harvest from a Sample Produced in, for Example, a Bioreactor and Concentrate (i.e., 10-Fold) by Centrifugation

ALVAC HIV was grown in the avian cell line EB14/074 in a bioreactor (e.g., a 10 L-bioreactor). The culture was harvested and aliquoted into 1 L sterile centrifuge bottles (700 mL/bottle), and centrifuged at 4000×g for 40 min at 4° C. using a Jouan KR422 centrifuge. The supernatant was discarded and the cells resuspended in 50 mL of 10 mM Tris-HCl pH 7.0-9.0 (per bottle). The mixture was vortexed vigorously and transferred into a 1 L sterile Nalgene bottle. The final volume of the concentrated material was brought to 1/10 of the initial harvest volume with 10 mM Tris-HCl pH 7.0-9.0 to produce a 10-fold (10×) concentrated harvest. The concentrated harvest was stored in a −80° C. freezer until further use.

### 2. Release Intracellular Poxvirus by an Appropriate Method Such as Cell Disruption by Direct Sonication to Produce a Crude Poxvirus Preparation.

The sonicator with associated inlet/outlet tubing was autoclaved. The Easyload II Masterflex pump was connected to the inlet line of the sonicator. The sonicator was equilibrated and associated lines by pumping 200 mL of 10 mM Tris-HCl pH 7.0-9.0 buffer at 50 mL/min flow rate. The 10× concentrated harvest was pumped through the sonicator at 50 mL/min flow rate. When sample reached the sonicator inlet, the sonicator was started at a power output of 55-65 Watts. The sonicated harvest was then collected through the sonicator outlets into a sterile bottle. This is a crude poxvirus preparation.

3. Clarify the Crude Poxvirus Preparation Using, for Example, Sequential Filtration with 5 μm and 3 μm Depth Filters

The 5 μm/3 μm filters (PALL, BY050P6 and BY030P6) set with associated inlet/outlet tubing was autoclaved. The Easyload II Masterflex pump was connected to the inlet line of the 5 μm filter. The depth filters were equilibrated by pumping 200 mL of 10 mM Tris-HCl pH 7.0-9.0 at 200 mL/min pump flow rate. The sonicated harvest was diluted with an equal volume of 10 mM Tris-HCl pH 7.0-9.0 buffer. Up to 500 mL of diluted harvest was pumped through a set of 5 μm/3 μm depth filters (5 μm followed by 3μ filter) at 200 mL/min flow rate followed by a flow rate of 400 mL/min to collect remaining sample. The depth filters were rinsed with 50 mL of 10 mM Tris-HCl pH 7.0-9.0 to chase the hold-up sample. The clarified crude poxvirus preparation was stored in a −80° C. freezer until further use.

4. Degrade Free DNA Present within the Clarified Crude Poxvirus Preparation Using a Reagent Such as Benzonase Nuclease.

Benzonase Nuclease was added to a pre-selected amount of clarified poxvirus preparation to a final concentration of 10-50 Units/ml. MgCl₂ (nuclease catalyst) was added to a final concentration of 2.0 mM. The components were mixed at 20±3° C. for 1 to 2 hours (depending on the particular preparation) in a mixing vessel with a magnetic stir bar. At the end of the digestion, EDTA was added at a final concentration of 5 mM to stop the enzyme reaction.

### 5. Produce a Semi-Purified Poxvirus Preparation by Gel Filtration Using an Appropriate Chromatographic Matrix and Buffer System Such as Sepharose 4 FF/6FF.

A column, adaptor and its associated tubing was sanitized overnight by filling the column with 1M NaOH. The NaOH was then drained and the column, adaptor and associated lines rinsed with 2-column volume of water for injection (WFI) followed by sanitization with 1-column volume of 70% ethanol. The column was then filled with 10 cm of WFI or equilibrating buffer and the desired volume of resin (Sepharose 4 FF or Sepharose 6 FF) poured into it to pack a 20 cm height column. WFI was mixed with the Sepharose 4FF or Sepharose 6FF media to create homogeneous solution. The top adaptor was positioned 3-10 cm above the surface of the liquid using the height adjuster handle. The top adaptor inlet tubing was attached to the AKTA Explorer system and 70% ethanol was pumped through it to sanitize the lines and wet the column nets to eliminate any trapped air using AKTA system. The resin was allowed to settle until a top clear liquid layer of 1-2 cm was visible. The top adaptor was lowered to 1 to 2 cm below the clear liquid layer and the adaptor O-ring sealed. The column outlet line was attached to the AKTA Explorer system. To pack the column, either WFI or equilibration buffer was pumped at 23-30 cm/hr using AKTA system. When the resin was packed to approximately 20 cm height, the top adaptor was lowered to approximately 0.5 cm above the settled resin bed and the adaptor O-ring sealed by turning the seal adjuster knob clockwise.

The AKTA explorer system was adjusted to bypass all the valves to reduce the back pressure at high flow rates. The sample line was sanitized in manual mode with 100 mL of 70% EtOH followed by rinsing with 200 mL of WFI and equilibrating with 100 mL of 10 mM Tris-HCl pH 7.0-9.0. The column was packed as described above and the resin equilibrated with 2-column volume of buffer (10 mM Tris-HCl pH 7.0-9.0) at 15-23 cm/hr until the curves of all process parameters (conductivity and pH) were stable. The AKTA sample line was placed into the clarified poxvirus preparation to be loaded onto the column inside a biocontainment cabinet. Sample loading volume was 15-20% of the column volume.

BPG100 (1.5 L Sepharose 4FF or 6FF) chromatography was run under a pre-programmed method having the following parameters:
Flow rate at 15 cm/hr
Equilibration with 50 mL of 10 mM Tris-HCl pH 7.0-9.0
Sample loading volume: 15% of the column volume
Elution with 2-column volume of 10 mM Tris-HCl pH 7.0-9.0
   The first peak eluted was found to contain 70-90% of virus (500 mL) was collected into a 500 ml sterile Nalgene bottle and this semi-purified poxviral preparation stored at 4° C. until further use.

### 6. Purify a Substantially Purified, Essentially Purified, or Purified Poxvirus Preparation Using an Appropriate Ion Exchange Matrix Such as Sepharose 4 FF (ANX)

An appropriate volume (dry resin volume equal to the volume of gel filtration virus-containing fraction) of ANX Sepharose 4 FF (GE Healthcare, Cat# 17-1287-01 and 171287-04) resin slurry (in 20% ethanol) was poured into a 2 L Nalgene bottle (containing magnetic stirrer) and the resin allowed to settle. Ethanol was removed by pumping at a flow rate of 200 ml/min using a Masterflex pump. The resin was washed twice with 2-resin volume of WFI followed by equilibration (twice) with 2-resin volume of 10 mM Tris-HCl pH 7.0-9.0. The resin was allowed to settle and the buffer removed by pumping at a rate of at 200-500 ml/min. An equal volume of sample was added to the settled resin and mix for 1 hr at 20±3° C. The resin was allowed to settle and unbound sample removed by pumping at a pumping rate of 200-500 ml/min. The resin was then washed twice with 2-resin volume of 10 mM Tris-HCl pH 7.0-9.0. The resin was then allowed to settle and the resulting wash sample removed by pumping at a pumping rate of 200-500 ml/min. The virus was eluted with 2-resin volume of 10 mM Tris-HCl pH 7.0-9.0/1M NaCl three times to produce a purified poxvirus preparation. The resin was then allowed to settle and the eluate removed by pumping at flow rate of 200-500 ml/min into a sterile bottle. Residual resin was removed from the elution pool using a 54 μm filter (Millipore polygard CN optical XL5) at a pump rate of 500-1000 mL/min.

### 7. Concentrate and Exchange Buffers by Filtration (i.e., Tangential Flow Filtration (TFF)).

The inlet (feed) line of TFF cartridge was connected with associated tubing to Masterflex pump and clamped one of the permeate outlets. Seventy percent was pumped ethanol through the cartridge and soaked the cartridge and associated lines overnight to dissolve storage glycerol and sanitized the system. The cartridge was rinsed with 10-12 L of WFI at pump rate of 200 mL/min, transmembrane pressure (TMP) of 0.2-0.4 bar, to remove ethanol and test for water flux. A clean water flux test was performed by measuring the permeate flow rate and TMP:

Flux [Liter, square meter, hour (LMH)/bar]={[permeate flow rate (mL/min)/cartridge area (m²)]×0.06}/TMP (bar)

The Flux should be greater than 399 LMH/bar for a new cartridge as indicated on certificate of analysis. The cartridge was equilibrated by circulating 0.5-1 L of 10 mM Tris-HCl pH 7.0-9.0 at cross flow rate of 200 mL/min for 30 min by clamping the permeate line. The sample was concentrated to 1/10 to ⅓ of the starting volume of the elution pool at shear rate of 8000-10000 sec⁻¹ and TMP at 0.4-1 bar. A buffer exchange was performed by continuous diafiltration with 3-volume of 10 mM Tris-HCl pH 7.0-9.0. The diafiltered sample was concentrated to desired volume. The permeate line was clamped and circulated the concentrate for 5-10 min at the above shear rate. The volume of the concentrated sample was collected and measured. The system was washed by pumping 200 mL of 10 mM Tris-HCl pH 7.0-9.0 at the above shear rate and the wash collected. The system was sanitized by passing 1 L of 70% ethanol.

A summary of this embodiment is shown below:

TABLE 1

| Process step | Parameters | Functions |
|---|---|---|
| Concentration of | 4000 g/4° C./40 min | Volume reduction for |
| harvest using | | column chromatography |
| centrifugation | | step |
| | | Buffer exchange |
| | | Partial removal of |
| | | macromolecules |
| Sonication to release | Power output 55-70 | Release of intracellular |
| virus using continuous | W at flow rate of 50 | virus |
| flow sonication | ml/min | |
| Clarification with | 5 μm and 3 μm | Removal of cell |
| depth filtration | depth filtration at | fragments |
| | pump rate of 200 | prior to column |
| | ml/min | chromatography |
| Degradation of DNA | 10-50 U/ml/20° C. ± | Facilitated DNA removal |
| with Benzonase | 3° C./1-2 h, followed | by subsequent process |
| Nuclease | by 5 mM EDTA | steps |
| | inactivation | |
| Purification using Gel | BPG100/500 | Removal of |
| filtration | column with height | macromolecules |
| chromatography | of 20 cm, 15% CV | Partial removal of very |
| | loading and linear | small particles |
| | velocity of 14.5 | Removal of residual |
| | cm/h, two | Benzonase |
| | consecutive runs | |
| | using the same | |
| | column | |
| Purification using | Performed in 5-18 L | Further removal of |
| ANX ion exchange | spinner flask or | impurities, soluble and |
| batch adsorption | stirring tank, eluted | particulates |
| | with 10 mM Tris- | |
| | HCl pH 7-9.0/1M | |
| | NaCl three times | |
| Concentration, | Performed using | Volume reduction |
| dilafiltration and | hollow fiber with | Buffer exchange |
| purification using TFF | lumen ID of 0.5-1 | Further removal of |
| | mm, length of 30-60 | impurities |
| | cm under shear rate | |
| | of 8,000-10,000 sec⁻¹; | |
| | concentrate 3-to | |
| | 10-fold prior to | |
| | diafiltration; TMP <1 | |
| | bar | |

### 8. DNA Extraction, Gel Electrophoresis, and Analysis

DNA extraction was performed essentially as described using Qiagen QIAamp DNA Blood Mini kit. Exceptions to the basic instructions include:
1. The Qiagen DNeasy Tissue kit (50) (Cat #69504) was utilized;
2. 2-Mercaptoethanol was not used in the Tissue lysis step comprising ATL buffer, Proteinase K and 2-Mercaptoethanol (as per SOP) and starting material sample;
3. Starting sample size was 200 μl (SM);
4. Sample was centrifuged at 13,200 rpm (instead of 14,000 rpm) in the 2nd wash step.

DNA gel electrophoresis was performed by preparing a 1.2% agarose gel (100 ml) by placing 1.2 g of agarose into a 250 mL conical flask; adding 100 mL of 1×TAE, and swirling to mix; microwaving the mixture for 1.5 min to dissolve the agarose; allowing the heated mixture to cool for ˜5 min down to about 60° C.; adding 10 μl of Ethidium Bromide and swirling to mix; pouring the agarose solution slowly into the tank, and inserting the comb; allowing the gel to solidify for 30 min; and, pouring 1×TAE running buffer into the gel tank to submerge the gel to 2-5 mm depth. Electrophoresis was performed by transferring an appropriate amount (18 μl) of each DNA sample into a new microfuge tube; adding an appropriate amount of 10× Loading buffer (2 μl) into each tube; loading the samples, and running the gel at 75 V for ˜40 min. The gel is then photographed under UV light to observe the samples.

DNA in viral starting material and purified products was determined by Quant-iT PicoGreen dsDNA assay kit (Invitrogen). With respect to the basic kit instructions, the only exception is that the DNA extracted from the crude samples is diluted 1:5 prior to serial dilution in the plate.

9. Total protein quantification using MicroBradford assay
1. Seven dilutions of protein standard BSA in PBS were prepared as representatives of the protein solutions to be tested. The range of BSA in this microtiter plate assay was 2.5-20 μg/well, using BSA stock of 250 μg/mL. Protein solutions were assayed in duplicates.
2. The appropriate volume of each sample was loaded in duplicate into adjacent microtiter plate wells, so that the protein content in each well fell within the standard curve.
3. An appropriate volume of PBS was added into each of the wells to a total volume of 200 μL. Fifty μL concentrated dye reagent was added into each sample well. The sample and reagent were mixed thoroughly using a multichannel pipetter.
4. The plate was incubated at RT for 15 min.
5. Absorbance was measured at 595 nm on a Dynex plate reader, using CurveEX regression.

### 10. Avian Protein Quantification Using ELISA

1. A microtiter plate plate was coated with 100 μl of anti-EB 14 antibody at 5 μg/ml, and incubated for 18 hr at RT in 0.05 M Na₂CO₃/NaHCO₃, pH 9.6.
2. The plate was blocked with 300 μl of 5% BSA/PBS and incubate at RT for 1 hr followed by two washes with 0.1% BSA/PBS/0.1% Tween20.
3. 100 μl of antigen diluted in 0.1% BSA/PBS/0.1% Tween20 was added, followed by incubation at RT for 1 hr, followed by 5 washes with 0.1% BSA/PBS/0.1% Tween20.
4. 100 μl of biotin-anti-EB14 antibody at 0.4/ml in 0.1% BSA/PBS/0.1% Tween20 was added, followed by incubation at RT for 1 hr followed by 5 washes with 0.1% BSA/PBS/0.1% Tween20.
5. 100 μl of avidin-HRP diluted 1/20000 in 0.1% BSA/PBS/0.1% Tween20 was added, followed by incubation at RT for 1 hr followed by 5 washes with 0.1% BSA/PBS/0.1% Tween20.
6. 100 μl of TMB/H₂O₂ (1:9) was added and incubated at RT for 10 min, and the reaction stopped with 50 μl of 1M H₂SO₄.
7. Absorbance was measured at 450 nm using Dynex plate reader.
   11. ALVAC Quantitative PCR (qPCR) and Avian qPCR

Quantification of ALVAC DNA and genomic equivalence (GEQ) was performed using ALVAC-specific quantitative PCR. For details, refer QO SOP New: Quantification of ALVAC DNA using Quantitative PCR. Avian qPCR is being developed in AvP France.

### 12. Benzonase ELISA

1. Two different ranges of six dilutions of Benzonase Endonuclease standard provided by
EMD ELISA kit were prepared. The ranges were 0.1-100 ng/mL, using Benzonase stock of 5 μg/mL. Samples were assayed in duplicates. The standards were loaded onto the plate with buffer 1 diluted such that the volume of each well was 100 μl.
2. 100 μl of each sample solution was loaded into separate microtiter plate wells.
3. 100 μl of buffer 1 was added into each Blank well.
4. The samples were incubated at RT for 2 hr.
5. The plate was emptied by inversion over towel paper, with repeated tapping of the plate many times to ensure complete removal of liquid. The wells were then filled with buffer 1, and incubated for 1 min. before emptying again. Step 5 was repeated three times.
6. The samples were incubated with 100 μl of reagent B diluted 1:100 with buffer 1 from the stock reagent B (horse radish peroxidase conjugated antibody) for 1 hr at RT.
7. The plate was then washed as described in step 6.
8. 60 μl of Reagent C was added to each well followed by incubation for 15 min (plate should be protected from light during incubation).
9. The enzymatic reaction was stopped by adding 140 μl Stop reagent (0.2M H₂SO₄) to each well.
10. The absorbance of each well was then read at 450 nm using Dynex plate reader.

### 13. Virus Titration Using CCID₅₀ Assay

ALVAC virus titres were measured by CCID₅₀ assay using QT35 cells. For details, refer SOP# 22PD-039 version 4.0. Exception: antibiotics in infection media were used twice as much as described in the SOP to eliminate contamination in CCID₅₀ assay due to sample exposure to open system during purification process. Test samples were sonicated indirectly.

### 14. Results

The procedure described above provides a composition with impurity (such as including but not limited to avian DNA and/or non-vector proteins) removal of greater than 90% (a purified preparation). Inthree embodiments, (Table 2), the overall virus recovery from the purification process was 20-52%. The clarification step removed 55-71% of total proteins. The subsequent gel filtration step removed an additional 61-72% of total proteins. Furthermore, the ANX ion exchange batch adsorption step removed 68-78%, followed by the TFF step which removed an additional 33-41% of total protein from the materials obtained from batch adsorption. As a result, the overall removal of total protein was approximately 97.6-98.2%. The avian proteins in the final purified products were removed by 98-99%. The ratio of total protein (pg) to CCID₅₀ was 11 to 17 (Table 2).

The degradation and removal of free avian DNA were also found to be effective through the purification process. Following the Benzonase treatment and gel filtration, only 1-1.5% of the avian DNA was recovered from clarified materials (Table 2). In addition, only 2.7-14% of avian DNA was recovered following the TFF step, indicating that an additional DNA (85-97%) was removed by the TFF step following ANX ion exchange (Table 2). The avian DNA content in the final products was removed by 99% (Quant-iT Picogreen dsDNAassay kit, Invitrogen, Cat# P11496 using the manufacturer's instructions).

The residual Benzonase was tested in the samples from gel filtration, ANX batch adsorption purified materials as well as in the final purified products using Benzonase ELISA (Benzonase Endonuclease ELISA Kit, EMD Chemicals, Inc. Cat# 1.01681.0002) using the manufacturer's instructions. The data showed that in all tested samples, Benzonase was removed by the gel filtration step to a level below the detection limit (0.2 ng/ml).

TABLE 2
Results from Purification processes at 10-L scale - 3 definition runs

| Process Step | Run 1 | Run 2 | Run 3 | Mean |
|---|---|---|---|---|
| Crude Harvest | | | | |
| Volume (mL) | 10000 | 6600 | 9500 | |
| Viable Cell Density (10⁶ cells/mL) | 3.2 | 5 | 5.3 | 4.5 |
| Infectious titer (logCCID₅₀/mL) | 6.8 | 6.4 | 6.5 | 6.6 |
| Total protein concentration (μg/mL) | 700 | 810 | 933 | 814 |
| Total proteins per dose (μg/dose) | 1032 | 3535 | 2950 | 2441 |
| Total proteins pg/CCID₅₀ | 104 | 352 | 295 | 250 |
| Avian proteins concentration (μg/mL) | 413 | 514 | 538 | 488 |
| Avian proteins per dose (μg/dose) | 685 | 2243 | 1150 | 1359 |
| Avian DNA concentration (ng/mL) | 1000 | 1600 | 1400 | 1333 |
| Avian DNA per dose (ng/dose) | 1584 | 6369 | 4427 | 4255 |
| Genomic equivalents per infectious particle | 828 | 6059 | 2062 | 2983 |
| 10-fold Concentration (centrifugation) | | | | |
| Virus recovery (%) | 69 | 90 | 100 | 86 |
| Sonication | | | | |
| Virus recovery (%) | 100 | 100 | 100 | 100 |
| Clarification | | | | |
| Virus recovery (%) | 60 | 100 | 100 | 87 |
| Total protein removal (%) | 65 | 71 | 55 | 64 |
| Avian DNA removal (%) | 14 | 13 | 20 | 16 |
| Benzonase treatment | | | | |
| Virus recovery (%) | 100 | 100 | 97 | 99 |
| Benzonase treatment plus gel filtration | | | | |
| Virus recovery (%) | 84 | 81 | 97 | 87 |
| Total protein removal (%) | 70 | 61 | 72 | 68 |
| Avian DNA removal (%) | 98.5 | 98.6 | 99 | 98.7 |
| ANX ion exchange batch adsorption | | | | |
| Virus recovery (%) | 80 | 70 | 100 | 83 |
| Total protein removal (%) | 69 | 78 | 68 | 72 |
| Avian DNA removal (%) | 16 | 38 | NA | 27 |
| TFF concentration | | | | |
| Virus recovery (%) | 66 | 60 | 65 | 63 |
| Total protein removal (%) | 37 | 33 | 41 | 37 |
| Avian DNA removal (%) | 97.3 | 86 | 92.4 | 92 |
| Purified bulk | | | | |
| Volume (mL) | 900 | 875 | 960 | |
| Virus recovery (%) | 20 | 52 | 40 | 37 |
| Infectious titer (logCCID₅₀/mL) | 7.2 | 7.0 | 7.1 | 7.1 |
| Total protein concentration (μg/mL) | 174 | 109 | 226 | 170 |
| Total proteins per dose (μg/dose) | 114 | 109 | 179 | 134 |
| Total protein removal (%) | 97.8 | 98.2 | 97.6 | 97.8 |
| Total proteins pg/CCID₅₀ | 11.5 | 11 | 17 | 13 |
| Avian proteins concentration (μg/mL) | 33 | 33 | 71 | 46 |
| Avian proteins per dose (μg/dose) | 20 | 33 | 56 | 36 |
| Avian proteins removal (%) | 99.3 | 99.2 | 98.7 | 99 |
| Avian DNA concentration (ng/mL) | 3.5 | 13 | 8.9 | 8.5 |
| Avian DNA removal (%) | 99.9 | 99.9 | 99.4 | 99.7 |
| Avian DNA per dose (ng/dose) | 2.2 | 13 | 8.9 | 7.4 |
| Genomic equivalents per infectious particle | 594 | 440 | 659 | 564 |

Note:
The recoveries of virus, protein or DNA listed at each step were the comparison with previous step.

### C. Purification of Alvac-Melanoma Vector

### 1. Materials

Materials used in the following studies include: QT35 cells; QT35 growth medium: SOP# 22PD-039; Ham's F-10 Medium (Gibco catalogue #11550-043); Medium 199 with Hank's Solution (Gibco catalogue #12350-039); Fetal Bovine Serum (FBS), JRH Cat. #12107-78P; Tryptose Phosphate Broth powder, (Difco, BD260300); Penicillin dihydrostreptomycin (Gibco); Benzonase Endonuclease, EM Industries, Inc. Cat# 1.01694.0002 and 1.1697.0002; Benzonase Endonuclease ELISA Kit, EMD Chemicals, Inc. Cat# 1.01681.0002; DNAeasy Kit, Qiagen, Cat# 69504; Quant-iT Picogreen dsDNAassay kit, Invitrogen, Cat# P11496; PBL Trypticase Soy Broth, Beckon Dickenson; Tryptic Soy Agar with 5% sheep blood (TSA II); ANX Sepharose 4 FF resin, Amersham Biosciences, Cat# 17-1287-01 and 171287-04; and, Sepharose 4 FF resin, Amersham Biosciences, Cat# 17-0149-01 and 17-0149-05.

### 2. Methods

a. Virus Release Using Sonication

ALVAC-Melanoma harvests were initially clarified using centrifugation (4000×g, 4° C. for 40 min) followed by filtration with 5 μM/3 μm depth filter as described above for the ALVAC-HIV virus. If frozen, virus samples were thawed in 37° C. water bath containing WFI water. Virus was sonicated before testing in CCID₅₀ assay. Samples were placed in 15 ml or 50 ml tubes and sonicated in the cup horn of the Virtis sonicator filled with chilled ice water for two 1 minutes with pulsing at 1 second on/1 second off and power output of 7.5. Samples were cooled on ice after sonication and the water temperature was monitored between sonications. A small amount of ice was added if necessary.

b. Virus Titration

ALVAC virus titres were measured by CCID₅₀ assay using QT35 cells. For details, refer to SOP# 22PD-039 version 4.0. Exception: antibiotics in infection media were used twice as much as described in the SOP to eliminate contamination in CCID₅₀ assay due to sample exposure to open system during purification process. Test samples were sonicated indirectly.

c. Electron Microscopy

Samples were examined using electron microscopy as described below:
1. The starting material was removed from the −80° C. freezer and thawed in a 37° C. waterbath;
2. The starting material was diluted 10-fold with 10 mM Tris-HCl, pH 8.0; 9.0; or 10.0 if necessary.
3. The samples were sonicated indirectly.
4. The samples were incubated either at RT for two hr or at 2-8° C. overnight when applied.
5. Following the appropriate incubation time, the virus was fixed using a fixing buffer containing paraformaldehyde and glutaraldehyde at 1:1 ratio of volume to the incubated viral suspension. Store the fixed viral samples at 2-8° C. until examination at Electron Microscopy Laboratory at University of Toronto.
6. The samples were prepared for examination in the transmission electron microscopy by negative staining, using the direct drop method. A drop of sample (5 μl) was placed directly onto a carbon-formvar coated 400 mesh copper grid. The sample was negatively stained by adding a drop (10 μl) of 2% phosphotungstic acid PTA (pH 6.5) or 2% uranyl acetate (UA) onto the prepared grid. After 30 second to a minute, the grid was blotted dry with filter paper. The samples were examined and photographed in a Hitachi H 7000 transmission electron microscope at 75 Kv.
   d. Benzonase Nuclease Degradation of Free Nucleic Acids (DNA)

The viral samples were thawed in a 37° C. water bath and sonicated indirectly as described in section 5.2.1. Desired amounts of the clarified materials were treated with various amount (U/ml) of benzonase at 20±3° C. for desired periods of time. MgCl₂ was added to a final concentration of 2.0 mM unless mentioned otherwise. The components were mixed with a stir bar and the suspension incubated according to the conditions specified. After the designated incubation time, the samples were maintained at −80° C. for further analysis.

e. DNA Extraction, Gel Electrophoresis, and Analysis

DNA extraction was performed essentially as described using Qiagen QIAamp DNA Blood Mini kit. Exceptions to the basic instructions include:
1. The Qiagen DNeasy Tissue kit (50) (Cat #69504) was utilized;
2. 2-Mercaptoethanol was not used in the Tissue lysis step comprising ATL buffer, Proteinase K and 2-Mercaptoethanol (as per SOP) and starting material sample;
3. Starting sample size was 200 μl (SM); and,
4. Sample was centrifuged at 13,200 rpm (instead of 14,000 rpm) in the 2nd wash step.

DNA gel electrophoresis was performed by preparing a 1.2% agarose gel (100 ml) by placing 1.2 g of agarose into a 250 mL conical flask; adding 100 mL of 1×TAE, and swirling to mix; microwaving the mixture for 1.5 min to dissolve the agarose; allowing the heated mixture to cool for ˜5 min down to about 60° C.; adding 10 μl of Ethidium Bromide and swirling to mix; pouring the agarose solution slowly into the tank, and inserting the comb; allowing the gel to solidify for 30 min; and, pouring 1×TAE running buffer into the gel tank to submerge the gel to 2-5 mm depth. Electrophoresis was performed by transferring an appropriate amount (18 μl) of each DNA sample into a new microfuge tube; adding an appropriate amount of 10× Loading buffer (2 μl) into each tube; loading the samples, and running the gel at 75 V for ˜40 min. The gel was then photographed under UV light to observe the samples. DNA in viral starting material and purified products was determined by PicoGreen assay (Molecular Probes, Eugene, Oreg.). With respect to the basic kit instructions, the only exception being that the DNA extracted from the crude samples was diluted 1:5 prior to serial dilution in the plate.

f. Total Protein Quantification Using MicroBradford Assay

As a standard, eight dilutions of a protein standard (BSA dissolved in PBS) were utilized as representative of the protein solutions to be tested. The range of BSA in this microtiter plate assay is 1.25-10.0 μg/well using low concentration samples and 10.0-60.0 μg/well for high concentration samples. A stock BSA solution (250 μg/mL) was used. Protein solutions were assayed in duplicates. An appropriate volume of each sample was loaded in duplicate into adjacent microtiter plate wells, so that the protein content in each well falls within the standard curve. An appropriate volume of PBS was added into each of the wells, such that the total volume is 200 and add 50 μL concentrated dye reagent into each sample well. The sample and reagent were mixed thoroughly using a multichannel pipetter (approximately ten times), incubated at RT for 15 minutes, and absorbance measured at 595 nm on Dynex plate reader, using CurveEX linear regression.

g. ANX Ion Exchange Batch Adsorption Chromatography

Resin was prepared as follows:
1. 625 mL of resin (500 mL dry resin) was poured into a 2 L Nalgene bottle and allowed to settle;
2. Ethanol was removed to the extent possible by pumping using Masterflex Digital Standard Drive and/or by pipette;
3. The resin was washed by adding two volumes (1000 mL) of WFI water and mixing for 10 min on stir plate. After settling, WFI was removed via pumping and/or pipette. This step was then repeated.
4. The resin was equilibrated using two volumes (1000 mL) of 10 mM Tris HCl, pH 7.4 and mixing for 10 min. After settling, 10 mM Tris HCl, pH 7.4 was removed via pumping and/or pipette. This step was then repeated.

Approximately 500 mL of test sample (i.e., ALVAC starting material) was combined with equilibrated resin and mixed for 60 min on stir plate. The mixture was then allowed to settle and unbound sample removed via pumping and/or pipette.

The mixture was then washed sample with two volumes (1000 mL) of 10 mM Tris HCl, pH 7.4 by mixing for 10 min. After settling, the wash sample was pumped out to a separate container. This was then repeated once.

Elution was accomplished by mixing the sample with 10 mM Tris pH 7.4/1M NaCl for 10 min. After settling, the elution sample was removed to a separate container by pumping or pipetting out. This was repeated twice more to yield combined filtered Elution Pool. The Elution Pool was then stored at −80° C. if possible, or 4° C.

h. Batch Adsorption Using Spinner Flask (10 L Scale)

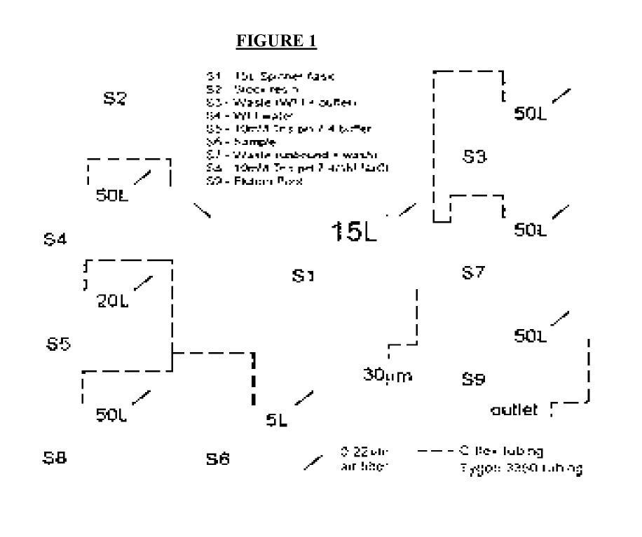

A batch adsorption system was set up as shown in FIG. 1. Resin was prepared as follows: ethanol was pumped out of 15 L spinner flask containing 6.25 L resin (5.0 L dry resin); the resin was washed using two volumes (10 L) of WFI water and mixing for 10 min; after settling, WFI was pumped out at 1 L/min, and this step was repeated once. The resin was then equilibrated using two volumes (10 L) of 10 mM Tris HCl, pH 7.4 and mixing for 10 min. After settling, 10 mM Tris HCl, pH 7.4 buffer was then pumped out at 1 L/min, and this step was repeated once. Five liters of sample was mixed with equilibrated resin for 60 min using stir plate. After settling, unbound sample was removed to separate container(s) by pumping at 750 mL/min. The sample was then washed with two volumes (10 L) of 10 mM Tris HCl, pH 7.4 by mixing for 10 min. After settling, the wash sample was pumped out at 1 L/min. This step was then repeated to yield combined wash ½ sample. Virus was eluted from the resin by mixing sample with 10 mM Tris HCl, pH 7.4/1M NaCl for 10 min. After settling, elution sample was removed to a separate container by pumping out at 1 L/min through a 30 μm filter. This step was repeated twice more to yield combined filtered Elution Pool. Eluted sample was stored at −80° C. if possible, or 4° C.

i. Packing a Large Scale BPG 100/200 Column (10 cm/20 cm Diameter)

Twenty-four size silicone tubing was connected to the bottom outlet of the BPG column for easier draining. The column, adaptor and associated tubings was sanitized by filling the column with 0.1M NaOH overnight. The NaOH was drained and the column rinsed with two-column volume of WFI. The column nets were wetted with 70% ethanol to eliminate trapped air. The column was filled with 10-15 cm of WFI or equilibrating buffer. The resin was shaken vigorously to make homogeneous media slurry. For every litre of packed column, 1.25 L or media slurry is pumped or poured. Thus, to pack a 20 cm height column, 1.5 L of packed resin is needed for BPG100 (10 cm diameter column) and 6.5 L for BPG 200 (20 cm diameter column). Homogeneous media slurry was poured into the column mixing it with WFI/equilibrating buffer. For a 1.5 L packed column bed, 1.88 L of media slurry was poured. For 6.5 L packed column bed, 8.13 L of media slurry was poured. The resin was allowed to settle until 1-2 cm of top clear liquid layer is visible. The bottom outlet was opened and the liquid slowly drained, making sure the top clear liquid layer is maintained. The adaptor was inserted and secured 3-10 cm above the surface of the liquid when the resin settled to the desired column height. The top adaptor inlet tubing was then connected to the AKTA Explorer system. Seventy percent ethanol was utilized to sanitize the lines and wet the column nets to eliminate any trapped air using AKTA system. The AKTA system pump was then stopped when the liquid started coming out from the top adaptor net. The adaptor was then lowered to approximately 0.5 cm above the settled resin bed, and the adaptor O-ring sealed by turning the seal adjuster knob clockwise. The 24-size silicone outlet tubing was replaced with AKTA compatible outlet tubing and connected to the AKTA system. The resin was equilibrated by pumping 2-CV of equilibrating buffer. 3 L of 10 mM Tris-HCl pH9/150 mM NaCl was then pumped at 20 mL/min for BPG 100 column and 13 L of 10 mM Tris-HCl pH9/150 mM NaCl at 80 mL/min for BPG 200 column.

j. Gel Filtration Chromatography at 2 L Bioreactor Scale (BPG 100)
1. The AKTA explorer system was adjusted to bypass all the valves to reduce back pressure at high flow rates.
2. The sample line (A15) was sanitized in manual mode with 100 mL of 70% EtOH, rinse with 200 mL of WFI and equilibrate with 100 mL of 10 mM Tris-HCl pH 9/150 mM NaCl using AKTA Explorer system. Collect waste using waste line in the biohood to sanitize and equilibrate that line.
3. The column was packed as described herein.
4. The BPG 100 packed column (1.5 L Seph 4FF) was connected to AKTA Explorer system.
5. The resin was equilibrated in manual mode with 2 CV (3.0 L) of 10 mM Tris-HCl pH 9.0/150 mM Tris-HCl buffer until the curves of all process parameters (conductivity and pH) were stable.
6. Inside a biohood, the sample line (A15) was inserted into the clarified harvest sample to be loaded onto the column; sample loading volume must be in the range of 12-18% of the column volume.
7. Chromatography was performed using a pre-programmed method:
   Starting Conditions: Flow 20 mL/min
   Equilibration with 50 mL of 10 mM Tris-HCl, pH 9/150 mM NaCl
   Load: 225 mL (15%) of the clarified harvest, benzonase treated
   Elution: 1800 mL of 10 mM Tris-HCl, pH 9/150 mM NaCl
   Sanitization: 1800 mL of 1M NaOH
   Rinse: 3000 mL of WFI
   Storage: 2000 mL of 20% EtOH
   Note: sanitization, rinse and storage steps are only required if the same resin will be reused in the future.
8. The first peak containing the virus (˜500 mL) is collected into 0.5 L sterile Nalgene bottle and store at 4° C. fridge until further use.
   k. Small-Scale TFF Using Minim System

Cartridge preparation was accomplished as follows:
1. The TFF cartridge, UFP-500-E-H22LA was connected onto the Minim System with one of the permeate outlets clamped.
2. The tubing and cartridge were flushed for 1 min with 70% ethanol at a TMP of not greater than 3 barg.
3. The permeate line was opened and flushing continued for approximately 10 min to dissolve glycerol and sanitize the cartridge.
4. The pump was stopped and all lines clamped. It was then allowed to sit overnight.
5. The system was flushed with WFI water for 1 min with permeate closed to remove ethanol and establish flow.
6. The permeate was opened and flushing continued for 5-10 min to remove residual ethanol.
7. A water flux test was conducted at minimum TMP (water flux must be equal or greater than indicated on the certificate of analysis (>=399 LMH/barg)).

Priming and equilibration was accomplished as follows:
1. 30 mL of media was circulated for 20 min at flow rate corresponding to desired shear rate.
2. The media was removed by flushing the system with 150-250 mL of 10 mM Tris HCl, pH 7.4 with permeate closed.
3. The permeate was opened and circulated with 10 mM Tris HCl, pH 7.4 for 20 min at flow rate corresponding to desired shear rate.

Sample concentration was accomplished as follows:
1. Sample was circulated for 1 min at a flow rate corresponding to desired shear rate with permeate closed to establish flow.
2. The permeate was opened to begin concentration, and collected in a separate waste container.

Diafiltration was accomplished as follows:
1. Once the desired concentration was reached, diafiltration was begun by adding one diafiltration volume to sample container.
2. When concentration was reached, step one was repeated twice more to complete three diafiltration volumes.
3. The sample was concentrated to almost zero volume, taking care not to allow air into the cartridge, whilst collecting retentate in separate container.
4. A sufficient volume of 10 mM Tris HCl, pH 7.4 buffer was added to the original sample container to dilute sample to correct concentration.
5. Sample was stored at −80° C.

The system was washed by passing approximate 25 mL of 10 mM Tris pH 7.4 buffer through system and collecting in separate Wash container and stored at 4° C. 200 mL 70% EtOH was run through cartridge to sanitize, and the cartridge discarded.

li. Small-Scale TFF Using AKTA Cross Flow System
1. A cross flow cartridge (UFP-500-C-H24U) was soaked in 25% EtOH overnight to ease in removal of storage glycerol.
2. The cross flow cartridge was rinsed with 1550 mL of WFI and equilibrate with 420 mL of 10 mM Tris-HCl pH 9.0 using pre-programmed method selected from Method Wizard followed by Preproduct steps in Method Editor window.
3. Water flux was checked using the rinsed cartridge by selecting Membrane System Evaluation followed by Normalized water flux in Evaluation window, making sure water flux is equal or greater than indicated on the certificate of analysis (>=399 LMH/barg). Additional rinsing was performed if the desired water flux was not reached.
4. Four hundred mL of ANX elution pool was concentrated to 40 mL and continuous diafiltration performed using 10 mM Tris-HCl pH 9.0 buffer. A pre-programmed method was selected in Method Wizard followed by Product steps in Method Editor Window.
5. Concentrated samples were then collected for testing.
6. When needed, TMP optimization or flux optimization was performed for each shear rate using TFF concentrated sample. A pre-programmed method was selected in Method Wizard followed by UF process optimization in Method Editor Window.
7. The optimal flux was determined from Flux vs. TMP graph generated in Membrane System Evaluation followed by Process Optimization in Evaluation window.
8. The cartridge was sanitized and AKTA cross flow system using pre-programmed method in Method Wizard followed by Post product steps in Method Editor window.
   m. Tangential Flow Filtration (TFF) at 2 L or 10 L Scale
1. TFF cartridges, UFP-500-C-3×2MA and UFP-500-C-6A, were connected to the Masterflex digital standard drive pump (Cole-Parmer Instrument Company, Model 77201-62, for 2-L scale) and Masterflex I/P Easy load pump (Cole-Parmer Instrument Company, Model 7529-10, for 10-L scale) with one (close to the feed side) of the permeate outlets clamped.
2. The cartridge was soaked with 70% ethanol overnight to dissolve glycerol and sanitize the cartridge at the same time.
3. The cartridge was rinsed with 10 L (2-L scale) or 100 L (10-L scale) of WFI at cross flow rate of 1 L/min and minimum TMP to get rid of ethanol.
4. The clean water flux test was performed by measuring permeate flow rate and TMP. Flux (lmh/bar)={[permeate flow rate (ml/min)/cartridge area (m2)]×0.06}/TMP (bar). For a new cartridge it should be >=399 lmh/barg according to certificate of analysis.
5. The cartridge was equilibrated by circulating 1 L (2-L scale) and 6 L (10-L scale) of 10 mM Tris-HCl pH 9.0/1.0M NaCI at cross flow rate of 1 L/min by clamping permeate for about 20 min.
6. The viral material from ion exchange batch absorption elution pool was pooled.
7. The sample was concentrated to ⅓ of the volume of the elution pool by increasing the feed flow rate gradually without any clamping on the retentate tubing, i.e. 1 L/min for 10 min., 1.5 L/min for 10 min, 2.1 L/min for 10 min and 4.3 L/min for the rest of concentration process. Permeate flow rate and feed pressure was measured.
8. An equal volume of 10 mM Tris-HCl pH 9.0 was added to the 3× concentrated sample to diafiltrate and concentrate to ⅓ of the starting volume.
9. Diafiltration was repeated three times.
10. The sample was further concentrated to approximately 100 mL (for 2-L scale) 500 mL (for 10-L scale).
11. The diafiltered concentrate was circulated with a bit higher feed flow for 5-10 min.
12. The concentrated sample was collected and measured.
13. The system was then washed by passing 200 mL (for 2-L scale) and 1 L (for 10-L scale) of 10 mM Tris-HCl pH 9.0.
14. The volume of wash sample was then collected.
15. The system was sanitized by passing 1 L of 70% ethanol.

### 3. Results

The purification process described herein includes the following steps: (a) concentration of crude harvest using centrifugation, (b) direct sonication to lyse cells, break up aggregates and release virus using sonitube, (c) depth filtration using 5 μm/3 μm filters to clarify material, (d) Benzonase treatment to degrade free DNA, (e) Sepharose 4 FF gel filtration chromatography to purify the virus and remove residual Benzonase, (f) ANX ion exchange batch adsorption to further purify the virus and (g) tangential flow filtration to purify and concentrate viral material and to exchange buffers. Each step of the process was evaluated for the DNA reduction of ALVAC melanoma produced in CEFs thereafter.

### A. Benzonase Digestion of Free Nucleic Acids (DNA)

The Benzonase concentration was defined as 50 U/mL with a reaction time of 2 hr at 20±3° C. for the degradation of free DNA in ALVAC HIV grown in EB14 (described above). These conditions were applied to the digestion of free DNA in three separate lots of ALVAC melanoma/CEFs (vCP1584, PX-06025, and PX-06026). The data showed that virus recovery from these preparations following Benzonase treatment varied from 23% to 79%, which were lower than that observed for ALVAC HIV/EB14. The result suggested that the Benzonase digestion conditions defined for ALVAC/EB14 should be modified for ALVAC/CEFs.

TABLE 3
Virus recovery after Benzonase digestion of free DNA

| Material | Digestion conditions | Virus recovery |
|---|---|---|
| vCP1548 | 50 U/ml, 2 hr RT | 24-76% |
| PX-06025 | 50 U/ml, 2 hr RT | 67% |
| PX-06026 | 50 U/ml, 2 hr RT | 79% |

The clarified materials were analyzed to determine virus titre and impurities. As shown in Table 4, the virus titre (log CCID₅₀) of the clarified ALVAC HIV produced in EB14 was between 6 to 7, and the CCID₅₀ to total DNA (pg) ratio was 0.14 to 1.4. The log CCID₅₀ of the clarified ALVAC melanoma produced in CEFs was 7.7 to 8.3. However, the ratios of titre to impurity in these samples were 11 to 64, 10-50 times higher than that of ALVAC HIV/EB14.

TABLE 4
Difference among clarified ALVAC harvests

| | Virus titre | Total | | | Virus recovery |
|---|---|---|---|---|---|
| Clarified | (logCCID₅₀/ | proteins | Total DNA | CCID₅₀/ | from Benzonase |
| harvest | ml) | (μg/ml) | (ng/ml) | DNA (pg) | digestion |
| ALVAC HIV/ | 6-7 | 700-1000 | 6949 | 0.14-1.4 | >90% |
| EB14 | | | | | |
| ALVAC | 7.7 | 536 | 780 | 64 | 24-76% |
| melanoma/ | | | | | |
| CEFs(vCP1548) | | | | | |
| ALVAC | 8.3 | 1159 | 6867 | 17 | 58% |
| melanoma/ | | | | | |
| CEFs (PX-06- | | | | | |
| 025) | | | | | |
| ALVAC | 8.1 | 1211 | 4193 | 11 | 60% |
| melanoma/ | | | | | |
| CEFs (PX-06- | | | | | |
| 025) | | | | | |

### B. Gel Filtration Chromatography

Gel filtration chromatography with conditions defined for ALVAC HIV/EB14 was next evaluated for purification of ALVAC melanoma/CEFs. The clarified sample (225 ml) was loaded on a 1.5 L (resin) column of 10 cm diameter with a flow rate of 20 ml/min. The virus recovery in 2 lots from the gel filtration was 84% and 87%, respectively, and the removal of total DNA was greater than 90% (Table 5). The data suggested that gel filtration chromatography with conditions defined for ALVAC/EB14 are suitable for purifying ALVAC melanoma/CEFs with similar virus yield and impurity removal.

TABLE 5
Virus yield and impurity removal from
gel filtration chromatography

| | % Sample | | | |
|---|---|---|---|---|
| | volume/resin | Virus recovery | Total protein | Total DNA |
| Material | volume | (%) | removal (%) | removal (%) |
| vCP1548 | 15% | 84 | 70 | Not tested |
| PX-06-025 | 15% | 87 | 65 | 94.8 |

### C. ANX Sepharose 4 FF Ion Exchange Batch Adsorption

ANX Sepharose 4 FF ion exchange batch adsorption with conditions defined for ALVAC HIV/EB14 was evaluated for the purification of ALVAC melanoma/CEFs. The fraction obtained from gel filtration was mixed with equal volume of ANX Sepharose 4 FF resin in 10 mM Tris-HCl, pH 9.0 buffer. The virus was eluted using 10 mM Tris-HCl, pH 9.0 containing 1 M NaCl. The virus recoveries from the two studies were 76% and 100%, respectively. The total protein measured by micro Bradford assay and total DNA measured by Picogreen assay were under the detection limit of the assays. Nevertheless, the ANX Sepharose 4 FF ion exchange batch adsorption with conditions defined for ALVAC HIV/EB14 can be used to purify ALVAC melanoma produced in CEFs.

TABLE 6
Virus yield from ANX ion exchange batch adsorption

| | Sample volume/resin | |
|---|---|---|
| Material | volume | Virus recovery (%) |
| vCP1548 | 1:1 | 76 |
| PX-06-025 | 1:1 | 100 |

### D. TFF to Concentrate and Exchange Buffers

TFF was used for concentrating the eluate from ANX ion exchange batch adsorption and for buffer exchange. When the TFF process developed for ALVAC HIV/EB14 was used to concentrate the eluate of ALVAC melanoma/CEFs, the virus recovery was 16-17% (Table 7), lower than that of ALVAC HIV/EB14. It was known that the virus titre (log CCID₅₀) of the eluate (the starting material for TFF) of ALVAC HIV/EB14 was 5 to 6 whereas that of ALVAC melanoma/CEFs was 6 to 7. However, the total protein level in the eluate of ALVAC HIV/EB14 was approximately 10 ug/ml whereas that of ALVAC melanoma/CEFs was under the detection limit of Bradford assay (1.25 ug/ml). Furthermore, the total protein concentration of the TFF concentrate from ALVAC melanoma/CEFs was 15.2-40 ug/ml, lower than that of ALVAC HIV/EB14 (109-226 ug/ml). Hence, the virus titre to impurity ratio was higher in ALVAC melanoma/CEFs, which could be the cause of extra loss of virus during the TFF process.

TABLE 7
Virus yield and total protein level in TFF concentrate

| | | Total protein concentration |
|---|---|---|
| Material | Virus recovery (%) | (ug/ml) |
| vCP1548 | 17 | 15.2 |
| PX-06-025 | 16 | 40 |

### E. Process Improvement and Re-Optimization for ALVAC Melanoma Produced in CEFs

### 1. Process Optimization of Benzonase Degradation of Free DNA

Various concentrations of Benzonase were tested for digestion of free DNA at room temperature for 2 hr. As shown in Table 8, when 10 U/ml of Benzonase was used, the total DNA was reduced by 4.2-fold. When Benzonase concentration was increased to 25 U/ml or 90 U/ml, the DNA reduction was only increased to 5.5- or 5.8-fold respectively, not as significant as that resulted from Benzonase increase from 0 U/ml to 10 U/ml. In addition, the highest virus recovery (77%) after Benzonase digestion was obtained when 10 U/ml Benzonase was used. Therefore, 10 U/ml of Benzonase was selected for digestion of free DNA in ALVAC produced in CEFs.

TABLE 8
DNA reduction and virus recovery after Benzonase treatment

| | | | | Virus |
|---|---|---|---|---|
| | | | Fold | recovery |
| | | | reduction | after |
| | Benzonase | Mean total | compared to | Benzonase |
| Material | (Unit/ml) | DNA (μg/ml) | untreated | digestion |
| PX-06-026 | 0 | 4.2 | | 100% |
| | 10 | 1 | 4.2 | 77% |
| | 25 | 0.77 | 5.4 | 55% |
| | 50 | 0.77 | 5.4 | 59% |
| | 75 | 0.76 | 5.5 | 60% |
| | 90 | 0.72 | 5.8 | 66% |

The digestion or treatment time was further evaluated for ALVAC melanoma/CEFs at 20±3° C. (RT). As shown in Table 9, at a Benzonase concentration of 25 U/ml, the level of DNA reduction was similar (6.4 to 6.8-fold reduction) among a range of treatment time, from 30 min to 120 min. The same held true for Benzonase treatment at 50 U/ml with a DNA reduction of 7.1 to 7.9-fold. These data suggested that Benzonase digestion of free DNA at room temperature for 30 min may be as effective as that for 2 hr. Based on the above results, the conditions of DNA digestion of ALVAC melanoma/CEFs was defined as 10 U/ml of Benzonase at 20±3° C. for 1 hr.

TABLE 9
DNA reduction after Benzonase treatment
for various periods of time

| | | | Fold |
|---|---|---|---|
| | | | reduction |
| Benzonase | Digestion | Mean total | compared to |
| (Unit/ml) | time (min) | DNA (μg/ml) | untreated |
| 0 | 30 | 6.4 | — |
| | 60 | 6.3 | — |
| | 120 | 5.4 | — |
| 25 | 30 | 1.0 | 6.4 |
| | 60 | 0.93 | 6.7 |
| | 120 | 0.79 | 6.8 |
| 50 | 30 | 0.81 | 7.9 |
| | 60 | 0.88 | 7.1 |
| | 120 | 0.74 | 7.2 |

### 2. Evaluation of 10 mM Tris-HCl, pH 7.4 in Purification Process

In the purification process developed for ALVAC HIV/EB14, 10 mM Tris-HCl, pH 9.0 was used in gel filtration and ANX ion exchange batch adsorption. Data from a stability study indicated that ALVAC appeared equally or more stable in 10 mM Tris-HCl, pH 7.4. To simplify the buffers usage in the purification process, virus recovery under the two pH conditions was compared.

Two purification runs were performed using the same starting material, clarified ALVAC melanoma/CEFs (lot# PX-06026), which was in 10 mM Tris-HCl, pH 7.4. The gel filtration step was performed using 10 mM Tris-HCl, pH 7.4 in both runs. 10 mM Tris-HCl pH 7.4/1 M NaCl and 10 mM Tris-HCl, pH 9.0/1 M NaCl were compared in ANX ion exchange batch adsorption and TFF in the two runs. The virus yields from gel filtration step from the two runs were 89% and 100% respectively, which were consistent with that using 10 mM Tris-HCl, pH 9.0. The virus recoveries in ion exchange and TFF steps using 10 mM Tris-HCl, pH 7.4 were close to that using 10 mM Tris-HCl, pH 9.0 (Table 10). The total DNA recoveries after three step-purification using 10 mM Tris-HCl, pH 7.4 were also similar to that using 10 mM Tris-HCl, pH 9.0. In conclusion, 10 mM Tris-HCl, pH 9.0 may be replaced with 10 mM Tris-HCl, pH 7.4 in all three steps of the purification process to achieve similar virus yield and total DNA removal.

TABLE 10
Comparison of virus and total DNA recovery using 10 mM Tris-HCl,
pH 9.0 and 10 mM Tris-HCl, pH 7.4 in the purification process

| | Virus recovery (% as compared to | | Total DNA recovery (% as compared to | |
|---|---|---|---|---|
| | previous step) | | clarified harvest) | |
| Purification | 10 mM Tris-HCl pH 9.0 | 10 mM Tris-HCl pH 7.4 | 10 mM Tris-HCl pH 9.0 | 10 mM Tris-HCl pH 7.4 |
| Gel filtration | >80% | 89-100% | n/a | 31% |
| ANX ion exchange batch | 77% | 68% | n/a | n/a |
| adsorption | | | | |
| TFF | 89% | 79% | 8.7% | 7.6% |

n/a, not available

### 3. Optimization of TFF Process

a. Evaluation of Adsorption of ALVAC to TFF Cartridge

The adsorption of ALVAC to the TFF membrane during the purification process was studied first in order to understand the potential mechanism underlying the low yield of the ALVAC melanoma/CEFs from the TFF step. The virus was circulated in the TFF system for various periods of time with the permeate port clumped. Hence no TMP was applied on the membrane and any virus loss should be caused by the adsorption of virus to the membrane or shear damage. Two shear rates were compared for virus loss during the TFF. It was found that the titre drop correlated with the length of circulation time, the longer the circulation, the greater drop of titre. After circulation of 30 min with a shear rate of 8000 sec-¹ or 12000-sec¹, a similar virus loss was observed (13% and 15% lost respectively), suggesting that the loss may be primarily caused by virus adsorption to the membrane. Moreover, when the virus was circulated for 2 hr, the higher the shear rate, the greater the virus loss, i.e. approximately 15% more virus loss at shear rate of 12000-sec¹ as compared to that of 8000 sec-¹ (Table 11). These results suggested that ALVAC may be adsorbed to the TFF membrane and higher shear could cause more product loss in a prolonged process. Therefore, the TFF process time should be as short as possible and the shear rate should be controlled between 8000 sec-¹ and 12000-sec¹ to minimize the loss of virus during TFF process.

TABLE 11
Virus loss when circulated in TFF cartridge

| | Virus titre logCCID50 | Virus titre logCCID50 | Virus titre logCCID50 | Virus titre logCCID50 |
|---|---|---|---|---|
| | when circulated | when circulated | when circulated | when circulated |
| | 0 min | 10 min | 30 min (recovery) | 2 hr (recovery) |
| Shear | 5.33 | 5.33 | 5.26 (85%) | 5.18 (70%) |
| 8000 sec−¹ | | | | |
| Shear | 5.33 | 5.34 | 5.27 (87%) | 5.06 (54%) |
| 12000 sec−¹ | | | | |

b. Determination of Optimal Operating Trans-Membrane Pressure (TMP) for Various Shear Rates

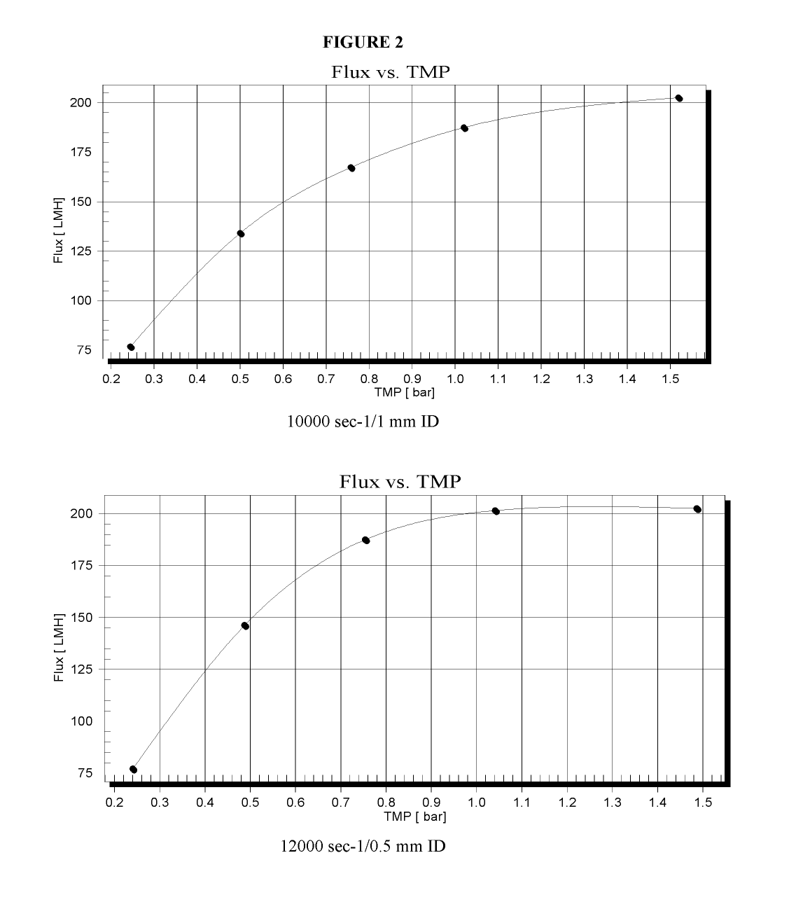

To establish an optimal operating TMP, the flux LMH (litre/meter²/hour) of TFF using two types of cartridges (lumen ID of 1 mm and 0.5 mm) was evaluated for various operating shear rates (Table 10). ANX ion exchange eluate of ALVAC melanoma/CEFs was used as material for TFF and cartridges of 38 cm² were used to perform the TFF experiments. The data showed that when cartridge of lumen ID 1 mm was used and the shear rate was 8000 sec⁻¹, the flux (LMH) reached a plateau when TMP was increased to 0.75 bar. When a higher shear rate 10000 was used, the flux plateaued later when TMP reached 1.5 bar (FIG. 2). Based on the linear range of the performance curves (FIG. 2), optimal operating TMP for shear rate of 8000, 10000 and 12000 were suggested as <0.5 bar, <0.75 bar and <0.75 bar, respectively. Similarly, the operating TMP for different shear rates using cartridges with lumen ID of 0.5 mm were suggested as <0.5 bar and <0.6 bar for shear rate of 8000 sec¹ and 12000 sec¹, respectively.

TABLE 12
Optimal operating TMP for various operating shear rates

| | ID 1 mm | | | ID 0.5 mm | | |
|---|---|---|---|---|---|---|
| | Shear rate (sec⁻¹) | | | | | |
| | 8000 | 10000 | 12000 | 8000 | 10000 | 12000 |
| TMP plateau | 0.75 | 1.5 | 1.5 | 1.0 | n/a | 1.0 |
| (bar) | | | | | | |
| Suggested | ≦0.5 | ≦1 | ≦1 | ≦0.75 | n/a | ≦0.75 |
| operating | | | | | | |
| TMP range | | | | | | |
| (bar) | | | | | | |

c. Evaluation of TFF Performance Under Different TMP and Shear Rates

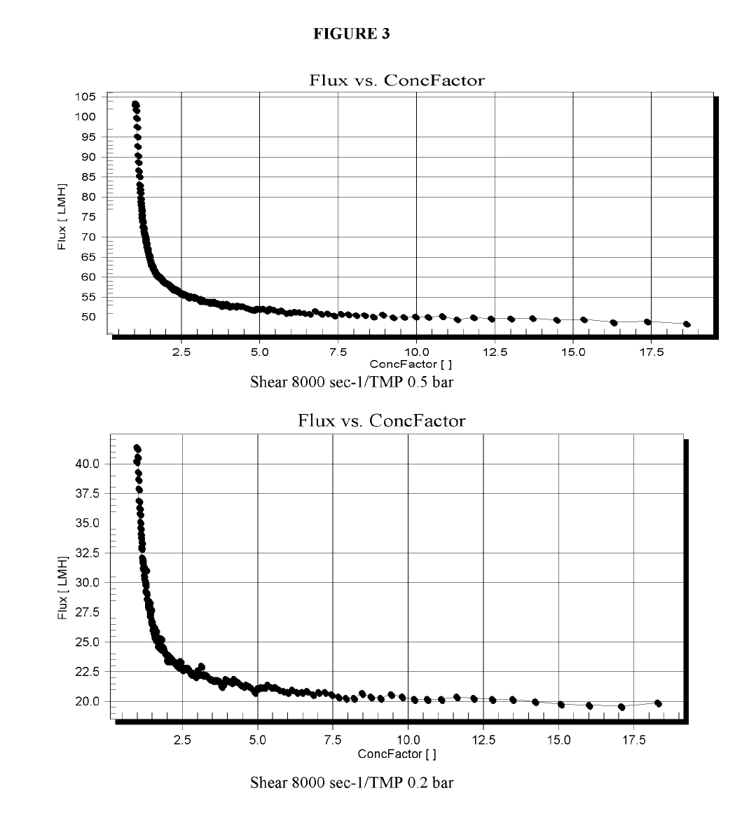

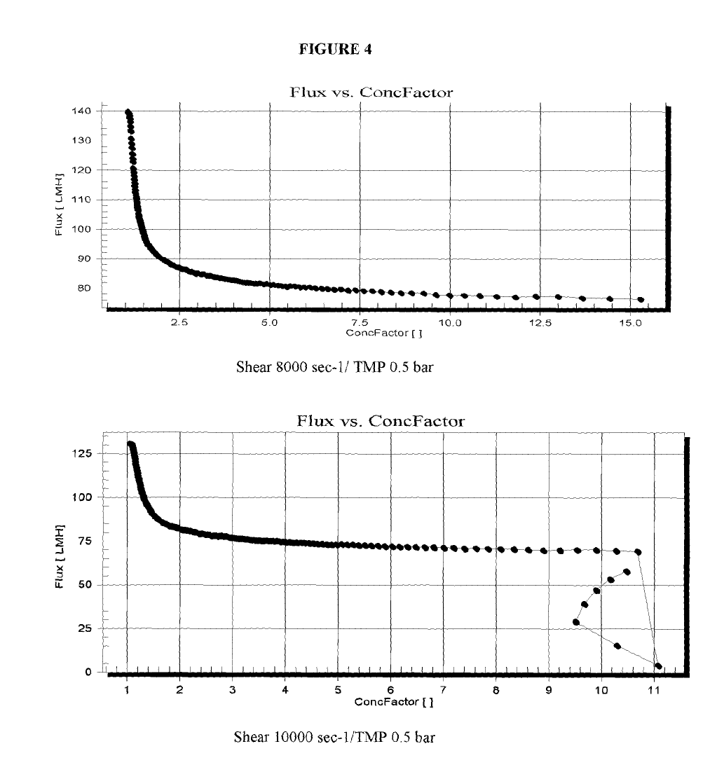

After the determination of optimal TFF operating ranges for different shear rates, the performance of TFF, i.e. the flux vs. concentration factor curve for a given shear rate and TMP was studied. When a shear rate of 8000 sec⁻¹ and TMP of 0.5 bar (optimal TMP range <0.75 bar) was used (for a cartridge with a lumen ID of 0.5 mm), the flux dropped from 105 LMH to 58 LMH (approximately 2-fold) when the sample was concentrated by 2-fold, indicating a membrane fouling at the starting of the concentration process. The poor TFF performance was suspected to be caused by a high TMP and therefore, a lower TMP, 0.2 bar was used in a later study. However, a similar flux drop was observed, suggesting that the lower TMP did not help to prevent membrane fouling (FIG. 3). A TFF cartridge with a lumen ID of 0.5 mm was also evaluated for performance under different shear rates (FIG. 4). Approximately 2-fold decrease of flux was observed when the sample was concentrated 2-fold at a shear rate of 8000 sec⁻¹ or 10000 sec⁻¹. These results suggest that membrane fouling occurs regardless of shear rate, TMP or lumen ID.

d. Evaluation of Membrane-Priming for Optimal Virus Recovery from TFF

It was understood from the above studies that ALVAC virus was adsorbed to the TFF membrane and membrane fouling occurs regardless of lumen id, TMP and shear rate. The next factor to examine was whether the membrane can be primed with certain reagents prior to the exposure of the membrane to virus to reduce the adsorption of the virus and membrane fouling. The media used for virus infection and the clarified ALVAC produced in CEFs were evaluated as priming reagents for TFF membrane. The above mentioned reagents were circulated in TFF for 20 min prior to introducing the viral material. The virus recovery from TFF with membrane primed with the media or the clarified viral material was similar to that from the TFF without priming (data not shown), suggesting that priming of TFF membrane did not increase the virus yield.

e. Purification of ALVAC Melanoma/CEFs

The ALVAC melanoma produced in CEFs vcp 2264 (lot# PX-06025) was purified using the modified purification process for ALVAC HIV/EB14. The virus recoveries from the purification steps including Benzonase digestion of free DNA, gel filtration chromatography, ANX ion exchange batch adsorption and TFF were 100%, 66%%, 100% and 40%, respectively. The virus yields from Benzonase treatment and ANX ion exchange were significantly improved upon process optimization. However, the virus recovery from the TFF step was only increased from 20% to 40%. Non-specific adsorption and membrane fouling may lead to poor performance of TFF. The overall virus yield was 28%. The removal of total proteins was 99% to a final concentration of 8.9 ug total proteins/dose. The removal of total DNA was 95.7% which resulted in a final DNA concentration of 172 ng/dose. Previous data from purification of ALVAC produced in EB14 showed that the average ratio of avian DNA to total DNA was 1.7% in the purified product. Assuming the same avian DNA to total DNA ratio, the avian DNA level in purified ALVAC melanoma/CEFs can be estimated at 2.9 ng/dose. (172 ng/ml×1.7%*10̂7/10̂7.29=2.9 ng/dose, assuming 10̂7 CCID₅₀ per 1 dose). The result from purification of ALVAC melanoma/CEFs were summarized in Table 13.

TABLE 13
Results from Purification of ALVAC/CEFs (2-L
scale) using modified purification process

| Process Step | Result |
|---|---|
| Clarified Harvest | |
| Volume (mL) | 225 |
| Infectious titer (logCCID₅₀/mL) | 7.98 |
| Total protein concentration (μg/mL) | 2565 |
| Total proteins per dose (μg/dose) | 256 |
| Total proteins pg/CCID₅₀ | 25.6 |
| Total DNA concentration (ng/mL) | 11317 |
| Total DNA per dose (ng/dose) | 1132 |
| Benzonase treatment (10 U/ml, RT, 2 | |
| hr) | |
| Virus recovery (%) | 147 |
| Benzonase treatment plus gel | |
| filtration | |
| Virus recovery (%) | 66 (compared to |
| | clarified) |
| Total protein removal (%) | 73 (compared to |
| | clarified) |
| Total DNA removal (%) | 86 (compared to |
| | clarified) |
| ANX ion exchange batch adsorption | |
| Virus recovery (%) | 100 (compared |
| | to previous step) |
| Total protein removal (%) | n/a |
| Total DNA removal (%) | 58 (compared to |
| | previous step) |
| TFF concentration | |
| Virus recovery (%) | 40 (compared to |
| | previous step) |
| Total protein removal (%) | n/a |
| Total DNA removal (%) | 56 (compared to |
| | previous step) |
| Purified bulk | |
| Volume (mL) | 200 |
| Virus recovery (%, overall) | 28 |
| Infectious titer (logCCID₅₀/mL) | 7.29 |
| Total protein concentration (μg/mL) | 17.5 |
| Total proteins per dose (μg/dose) | 8.9 |
| Total protein removal (%, overall) | 99% |
| Total proteins pg/CCID₅₀ | 0.9 |
| Total DNA concentration (ng/mL) | 338 |
| Total DNA removal (%, overall) | 95.7 |
| Total DNA per dose (ng/dose) | 172 |
| Estimated avian DNA (ng/dose) based | 2.9 |
| on ratio of avian DNA to total DNA | |
| from ALVAC/EB14 (average 1.7%) | |

f. Concentration of Clarified ALVAC Melanoma/CEF Using TFF

Clarified ALVAC melanoma/CEFs (to reach log CCID₅₀>8.5) was concentrated for a stability study. TFF was evaluated as a concentration approach, comparing two TFF systems (AKTA-cross flow and Minim TFF), different shear rates, and TMPs. It was found that the virus was recovered at 100% from all conditions tested with final tire log CCID₅₀ of 8.7-9.0. The removal of total proteins was 15-30% but 100% of total DNA was retained (Table 14). Hence, ALVAC harvest produced in CEFs can be concentrated using TFF to increase the titre/ml when the reduction of host cell DNA (from primary cells such as CEF) is not a major concern.

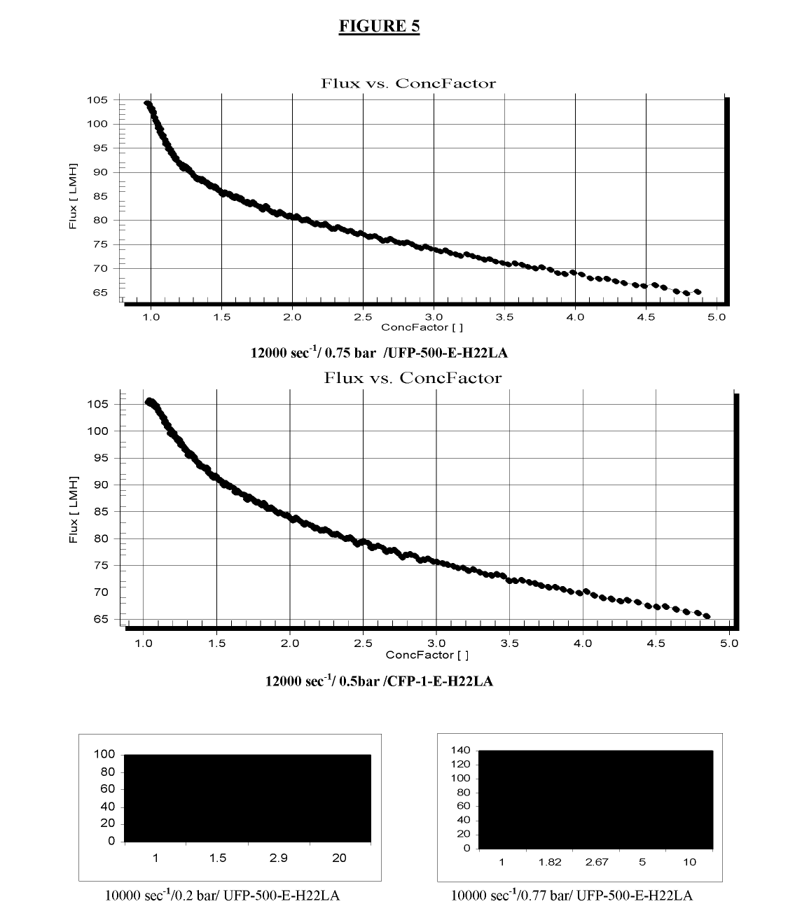

The TFF performance curve, i.e. flux vs. concentration factor curve, was then studied to understand the higher virus recovery from concentration of clarified material using TFF as compared to that from concentration of purified material. The performance curves (FIG. 5) showed that, at a shear rate of 12000 sec-¹, the flux dropped from 105 LMH to 85 LMH (approximately 1.2-fold) when the sample was concentrated by 2-fold. Similarly, at a shear rate of 10000 sec-¹, the flux dropped 1.2- to 1.3-fold when the sample was concentrated by 2-fold. In contrast, 2-fold decrease of flux was observed when the purified sample was concentrated 2-fold (FIGS. 3 and 4). These data suggest that a better TFF performance was obtained when concentrating clarified materials than purified materials. The lower virus to impurity ratio may contribute to the higher virus recovery from TFF.

TABLE 14
Results of concentration of ALVAC melanoma/CEFs using TFF

| | | | | | Total | |
|---|---|---|---|---|---|---|
| TFF | System | Shear rate | | Virus recovery | protein | Total DNA |
| cartridge | used | sec−¹ | TMP (bar) | (logCCID50) | recovery | recovery |
| CFP-1-E- | AKTA- | 12000 | 0.5 | 100% (8.66) | 63% | 100% |
| H22La | cross flow | | | | | |
| UFP-500-E- | AKTA- | 12000 | 0.75 | 100% (8.69) | 67% | 100% |
| H22LA | cross flow | | | | | |
| UFP-500-E- | Minim | 10000 | 0.76 | 100% (8.87) | 91% | 100% |
| H22LA | system | | | | | |
| UFP-500-E- | Minim | 10000 | 0.2 | 100% (9.0) | 84% | 100% |
| H22LA | system | | | | | |

A DNA reduction process developed for ALVAC melanoma/CEFs using the platform purification process for ALVAC/EB14 with process re-optimization is outlined in Table 15.

TABLE 15
Purification process for ALVAC grown in CEFs

| Process step | Parameters | Functions |
|---|---|---|
| Concentrate harvest | Centrifugation: 4000 | Volume reduction for |
| using centrifugation or | g/4° C./40 min | column chromatography |
| TFF | TFF: Performed | step |
| | using TFF hollow | Buffer exchange |
| | fiber with lumen ID | Partial removal of |
| | of 0.5 mm, length of | macromolecules |
| | 30-60 cm and shear | |
| | rate of 8000-12,000 | |
| | sec⁻¹; concentrate 5- | |
| | 10-fold prior to | |
| | diafiltration | |
| Sonicate cells to release | Power output 55-70 | Release of intracellular |
| virus using continuous | W at flow rate of 50 | virus |
| flow sonication | ml/min, sonicate | Break up viral aggregates |
| | twice | |
| Clarify virus with depth | 5 μm and 3 μm | Removal of cell fragments |
| filtration | depth filtration at | prior to column |
| | pump rate of 200 | chromatography |
| | ml/min | |
| Degrade free DNA with | 10 U/ml/20° C. ± 3° C./ | Facilitated DNA removal |
| Benzonase | 1 h, followed by 5 | by subsequent process |
| | mM EDTA | steps |
| | inactivation | |
| Purify virus using gel | BPG200/500 | Removal of |
| filtration | column with height | macromolecules |
| chromatography | of 20 cm, 15% CV | Partial removal of very |
| | loading and linear | small particles |
| | velocity of 14.5 | Removal of residual |
| | cm/h, two | Benzonase |
| | consecutive runs | |
| | using the same | |
| | column | |
| Purify virus using ANX | Performed in | Further removal of |
| ion exchange batch | spinner flask or | impurities, soluble and |
| adsorption | stirring tank, eluted | particulates |
| | with 10 mM Tris- | |
| | HCl pH 7-9.0/1M | |
| | NaCl three times | |
| Concentrate, dilafiltrate | Performed using | Volume reduction |
| and purify final product | TFF hollow fiber | Buffer exchange |
| using TFF | with lumen ID of 0.5 | Further removal of |
| | mm, length of 30-60 | impurities |
| | cm and shear rate of | |
| | 8000-12,000 sec⁻¹; | |
| | concentrate 5-10- | |
| | fold prior to | |
| | diafiltration; TMP <1 | |
| | bar | |
| Store purified products | | Storage of purified |
| | | products |

All references cited, listed, or otherwise referred to herein are incorporated by reference in their entirety into this disclosure. While a description of certain embodiments of the methods described herein, it is to be understood that variations thereof are contemplated.

### REFERENCES

Amersham Biosciences, “Selecting hollow fibres and cartridges and systems”, 2000.
Auricchio, et al. (2001) “Isolation of highly infectious and pure adeno-associated virus type 2 vectors with a single-step gravity-flow column” Hum. Gene Ther. 12, 71-76.
Bachman, et al. A simple for the rapid purification of copia virus-like particles from Drosophila Schneider 2 cells. Journal of Virological Methods 115 (2004): 159-165.
Blanche, et al. (2001) “Polypeptide composition of an adenovirus type 5 used in cancer gene therapy” J. Chromatogr. A 921, 39-48.
Braas, et al. (1996) “Strategies for the isolation and purification of retroviral vectors for gene therapy” Bioseparation 6, 211-228.
Bishop, et al. Rapid and efficient purification of hepatitis A virus from cell culture. Journal of Virological Methods, Vol. 47, Iss. 1-2 (1994): 203-216.
Branovic, et al., 2003, Application of short monolithic columns for improved detection of viruses, J. Virological Meth. 110: 2: 163-171.
Carlsson, et al. Purification of infectious pancreatic necrosis virus by anion-exchange chromatography increases the specific infectivity. Journal of Virological Methods. 47 (1994): 27-36.
Clark, et al. (1999) “Highly purified recombinant adeno-associated virus vectors are biologically active and free of detectable helper and wild-type viruses” Hum. Gene Ther. 10, 1031-1039.
Downing, et al. Active respiratory syncytial virus purified by ion-exchanged chromatography: characterization of binding and elution requirements. Journal of Virological Methods. 38 (1992): 215-228.
Dubois et al. Large-scale purification of inactivated hepatitis A virus by centrifugation in non-ionic gradients. Journal of Virological Methods Vol. 32, Iss. 2-3 (1991): 327-334.
Eveleth, et al. (2000) “Purifying the Sendai virus in one step using ion-exchange chromatography” BioPharm 1, 40-43.
Gao, et al. (2000) “Purification of recombinant adeno-associated virus vectors by column chromatography and its performance in vivo” Hum. Gene Ther. 11, 2079-2091.
GE HealthCare Data Sheet, Streamline Direct CST I
Hubble, J. (1997) “Affinity cell separation: problems and prospects” Trends Biotechnol. 15, 249-255.
Huyghe, et al. (1995) “Purification of a type 5 recombinant adenovirus encoding human p53 by column chromatography” Hum. Gene Ther. 6, 1403-1416.
Karger A. et al. Simple and rapid purification of alphaherpesviruses by chromatography on a cation exchange membrane. Journal of Virological Methods. 70 (1998) 219-224.
Kaludov, et al. A. (2002) “Scalable purification of adeno-associated virus type 2, 4, or 5 using ion exchange chromatography” Hum. Gene Ther. 13, 1235-1243.
Kuiper, et al. (2002) “Purification of a functional gene therapy vector derived from moloney murine leukaemia virus using membrane filtration and ceramic hydroxyapatite chromatography” Biotechnol. Bioeng. 80, 445-453.
Loa C. C. et al. Purification of turkey coronavirus by Sephacryl size-exclusion chromatography. Journal of Virological Methods. 104 (2002): 187-194.
Lyddiatt, A., and O'Sullivan, D. A. (1998) “Biochemical recovery and purification of gene therapy vectors” Curr. Opin. Biotechnol. 9, 177-185.
Ronald, et al. Comparison of methods for concentration and purification of bovine viral diarrhea virus. Journal of Virological Methods. Vol. 16, Iss. 4 (1987): 271-279.
Mbiguino, et al. Purification of human respiratory syncytial virus: superiority of sucrose gradient over percoll, renografin, and metrizamide gradients. Journal of Virological Methods Vol. 31, Iss. 2-3 (1991): 161-170.
Merck KgaA technical information “Fractogel EMD process media virus isolation”.
Merck KgaA technical information, “Benzonase Nuclease”.
Millipore Data Sheet “Affnity chromatography media Matrex Cellufine™ Sulfate: For concentration, purification and depyrogenation of virus, viral/microbial antigens, heparin binding proteins”.
Millipore Data Sheet, “PreoSep-PB Chromatography Media”
Monolith Summer School, Jun. 6-9, 2004, Applications in Biochromatography, Bioconversion and solid phase synthesis, Book of Abstracts, Portoroz, Slovenia
Njayou, et al. Purification of measles virus by affinity chromatography and by ultracentrifugation: a comparative study. Journal of Virological Methods. Vol. 32, Iss. 1 (1991): 67-77.
O'Keeffe, et al. (1999) “The affinity adsorptive recovery of an infectious herpes simplex virus vaccine” Biotechnol. Bioeng. 62, 537-545.
O'Neil, et al. (1993) “Virus harvesting and affinity-based liquid chromatography: A method for virus concentration and purification” Bio/Technology 11, 173-178.
Prior CP et al., 1996, Inactivation and purification of human immunodeficiency virus-1 as antigen for the treatment of HIV-1 infection, BioPharm, 10: 22-34
Sartorius technical information “Rapid purification of alphaherpesviruses”
Sephacryl® High Resolution Instructions, Amersham Biosciences.
Snyder, et al. (2002) “Production of clinical-grade recombinant adeno-associated virus vectors” Curr. Opin. Biotechnol. 13, 418-423.
Stancar A. Monolithic Support for Chromatography, 2003, Genetic Eng. News, 23: (19), MaryAnne Liebert, Inc. Publishers.
Summerford, et al. (1999) “Viral receptors and vector purification: New approaches for generating clinical-grade reagents” Nat. Med. 5, 587-588.
Tamayose, et al. (1996) “A new strategy for large-scale preparation of high-titer recombinant adeno-associated virus vectors by using packing cell lines and sulfonated cellulose column chromatography” Hum. Gene Ther. 7, 507-513.
Transfiguracion, et al. 2003. Size-exclusion chromatography purification of high-titre vesicular stomatitis virus G glycoprotein-pseudotyped retrovectors for cell and gene therapy applications, Human Gene Therapy, 14:1139-1153.
Turnbull, A. E., Skulimowski, A., Smythe, J. A., and Alexander, I. E. (2000) “Adeno-associated virus vectors show variable dependence on divalent cations for thermostability: Implications for purification and handling” Hum. Gene Ther. 11, 629-635.
Villegas, et al. A rapid method to produce high yields of purified rotavirus particles. Journal of Virological Methods 104 (2002): 9-19.
Zolotukhin, et al. (1999) “Recombinant adeno-associated virus purification using novel methods improves infectious titer and yield” Gene Ther. 6, 973-985.
U.S. Pat. No. 6,146,874
U.S. Pat. No. 6,410,300
U.S. Pat. No. 5,719,049 U.S. Pat. No. 6,593,123

1-17. (canceled)

18. A process for purifying poxvirus from cell culture consisting of the steps of: a) harvesting poxvirus-containing cells; b) disrupting the cells to produce a crude poxvirus preparation; c) submitting the crude poxvirus preparation to gel filtration to produce a semi-purified poxvirus preparation; and, d) submitting the semi-purified poxvirus preparation to anion exchange chromatography to produce a purified poxvirus preparation.

19. The process of claim 18 consisting of the steps of:
a) lysing cells infected with a poxvirus to produce a crude poxvirus preparation;
b) submitting the crude poxvirus preparation obtained in step a) to gel filtration on Sepharose 4 Fast Flow or Sepharose 6 Fast Flow resin equilibrated with 10 mM Tris-HCl, pH 7.0-9.0 to produce a semi-purified poxvirus preparation;
c) submitting the semi-purified poxvirus preparation obtained in step b) to anion exchange chromatography on ANX Sepharose 4 Fast Flow resin equilibrated with 10 mM Tris-HCl, pH 7.0-9.0 such that poxvirus is adsorbed to the resin; and,
d) eluting the poxvirus adsorbed in step d) employing 10 mM Tris-HCl, pH 7.0-9.0/1M NaCl.

20. The method of claim 19 wherein the crude poxvirus preparation is clarified prior to performing step b).

21. A method of purifying recombinant poxvirus virions from contaminants, comprising:
(a) introducing a poxvirus vector into a suitable host cell;
(b) culturing the host cell to produce poxvirus virions;
(c) preparing a lysate from said host cells of step (b);
(d) passing the lysate over an anion exchange chromatography matrix, thereby binding said the recombinant poxvirus to anion exchange chromatography matrix; and,
(f) eluting the poxvirus from the anion exchange chromatography matrix.

22. The method of claim 21 wherein the lysate of step (c) is prepared by sonicating and the lysate is treated with a nuclease prior to performing step (d).

23. The method of claim 21 wherein the lysate of step (c) is subjected to gel filtration chromatography prior to performing step (d).

24. The method of claim 23 wherein the lysate of step (c) is prepared by sonicating and the lysate is treated with a nuclease prior to performing gel filtration.

25. The method of claim 21, wherein the ion exchange matrix is selected from the group consisting of a strong anion exchanger, a weak anion exchanger, a strong cation exchanger, and a weak cation exchanger.

26. The method of claim 21 wherein the ion exchange matrix is selected from the group consisting of Q Sepharose™ Fast Flow, SP Sepharose™ Fast Flow, CM Sepharose™ Fast Flow, DEAE Sepharose™ Fast Flow, and ANX Sepharose™ 4 Fast Flow.

27. The method of claim 21 wherein the ion exchange matrix is ANX Sepharose™ 4 Fast Flow.

28. A method for producing a purified poxvirus preparation comprising the steps of, in combination:
i. obtaining a poxvirus harvest from a cell culture sample;
ii. releasing intracellular poxvirus from cells contained in the sample to produce a crude poxvirus preparation;
iii. clarifying the crude poxvirus preparation by filtration;
iv. treating the preparation of step (c) with a nuclease;
v. subjecting the preparation of step (d) to gel filtration to produce a semi-purified poxvirus preparation;
vi. subjecting the semi-purified poxvirus preparation to ion exchange chromatography to produce a purified poxvirus preparation.

29. The method of claim 28 wherein step (f) utilizes an ion exchange matrix selected from the group consisting of a strong anion exchanger, a weak anion exchanger, a strong cation exchanger, and a weak cation exchanger.

30. The method of claim 28 wherein step (f) utilizes an ion exchange matrix selected from the group consisting of Q Sepharose™ Fast Flow, SP Sepharose™ Fast Flow, CM Sepharose™ Fast Flow, DEAE Sepharose™ Fast Flow, and ANX Sepharose™ 4 Fast Flow.

31. The method of claim 21 wherein step (f) utilizes the ion exchange matrix ANX Sepharose™ 4 Fast Flow.
